SEQUENCE LISTING (3 sequences)

SEQ ID NO: 1 - protein, 37 aa, Homo sapiens
LLGDFFRKSKEKIGKEFKRIVQRIKDFLRNLVPRTES

SEQ ID NO: 2 - protein, 18 aa, Unknown
  antimicrobial peptide C18G
ALYKKLLKKLLKSAKKLG

SEQ ID NO: 3 - protein, 11 aa, Artificial Sequence
  internally quenched fluorogenic peptide substrate for fluorometric assay
GKPILFFRLKX

(19) United States
(12) Patent Application Publication
(10) Pub. No.: US 2012/0064062 A1
(43) Pub. Date: Mar. 15, 2012

(54) INHIBITORS OF BACTERIAL PLASMINOGEN ACTIVATORS

(76) Inventors: Jon D. Goguen, Holden, MA (US); Daniel Aiello, Worcester, MA (US); Donald T. Moir, Concord, MA (US); Bing Li, Northborough, MA (US)

(21) Appl. No.: 13/231,769

(22) Filed: Sep. 13, 2011

Related U.S. Application Data
(60) Provisional application No. 61/382,370, filed on Sep. 13, 2010.

(51) Int. Cl. A61K 31/357; C07D 333/34; A61K 31/381; C07C 311/21; A61K 31/18; C07D 405/12; C07D 217/18; A61K 31/443; A61K 31/472; A61P 31/04; A61P 31/00; A61K 39/395; A61P 31/12; A61P 35/00; A61P 25/04; A61P 37/04; A61K 38/22; A61P 25/00; A61P 1/08; A61P 37/08; A61K 38/18; A61P 25/20; A61P 21/02; A61P 23/00; A61P 25/08; A61P 25/24; A61P 25/18; C07D 319/16

(52) U.S. Cl. 424/130.1; 549/366; 514/452; 549/65; 514/445; 564/87; 514/603; 546/284.1; 546/144; 514/337; 514/307; 514/9.7; 514/7.7

(57) ABSTRACT

Organic compounds showing the ability to inhibit bacterial omptin proteases, specifically Yersinia pestis plasminogen activator (Pla) are disclosed. The disclosed Y. pestis plasminogen activator inhibitor compounds are useful for treating, preventing, or reducing the spread of infections by Y. pestis.

### CROSS-REFERENCE TO PRIORITY APPLICATIONS

This application claims priority to U.S. Provisional Appln. No. 61/382,370 filed Sep. 13, 2010, the contents of which are incorporated herein.

### STATEMENT REGARDING FEDERALLY SPONSORED RESEARCH

The invention described herein was supported in part by NIH/NIAID grant no. AI-081399. Accordingly, the United States Government has certain rights in the invention.

### FIELD OF THE INVENTION

This invention is in the field of therapeutic drugs to treat bacterial infection and disease. In particular, the invention provides organic compounds that inhibit bacterial omptin proteases, specifically Yersinia pestis plasminogen activator (Pla).

### BACKGROUND OF THE INVENTION

Plague is caused by the Gram-negative bacterium, Yersinia pestis. Among the oldest documented infectious diseases, plague has caused multiple epidemics and at least three pandemics throughout recorded history. Plague usually manifests in humans in bubonic (infection of lymph nodes) or pneumonic (infection of lungs) forms, but may also spread to the blood resulting in a septicemic form of the disease. Bubonic plague typically results from the bite of a flea infected with Y. pestis bacteria, whereas pneumonic plague may be initiated by intimate contact and inhalation of contaminated nasal and airborne droplets from a patient or infected animal. The clinical presentation of bubonic plague is a very painful, usually swollen, hemorrhagic, necrotic, and often hot-to-the touch lymph node, called a bubo. Onset of bubonic plague is usually 2 to 6 days after a person is exposed to (infected with) the plague bacillus. The incubation period of primary pneumonic plague is 1 to 3 days and is characterized by development of an overwhelming pneumonia with high fever, cough, bloody sputum, and chills.

The mortality rates for plague are staggering. In untreated cases of bubonic plague there is a 40%-60% mortality rate, and in the case of pneumonic plague, the mortality is 100% for patients not treated within the first 24 hours of infection. A primary septicemic plague may also occur when the infecting plague bacillus bypasses the lymph nodes and proliferates in the circulatory system. If left untreated, the mortality rate of septicemic plague is 100%.

There were a total of 38,310 cases reported to the World Health Organization during the last documented 15-year period by 25 countries, with 2,845 deaths (Galimand, M., et al., Antimicrob. Agents Chemother., 50: 3233-6 (2006)). In the United States an average of approximately 10 to 20 cases of plague are reported annually. During the 1980s, epidemic plague occurred each year in Africa, Asia, or South America. Almost all of the cases reported during the decade occurred among people living in small rural towns, villages, or agricultural areas. In the early 1990s, outbreaks of plague also occurred in East African countries, Madagascar, Peru, and India (Dennis and Hughes, N. Eng. J. Med., 337(10): 702-704 (1997)). Plague epidemics are generally associated with human contact with rats carrying fleas infected with Y. pestis, although, other rodents infested with infected fleas may serve as reservoirs of the disease as well. For example, in the Southwestern United States, “sylvatic” plague may result from transmission of plague bacteria to humans by the bite of infected fleas populating a variety of rodents, including ground squirrels, prairie dogs, marmots, mice, and tree squirrels.

If administered sufficiently early, a number of antibiotics (e.g., streptomycin, chloramphenicol, tetracycline), alone or in combination, can be effective against plague. Antibiotics may also be administered prophylactically to any individual that is presumed to be at risk for plague, e.g., anyone suspected of contacting infected individuals or animals. However, reliance on treating plague solely with antibiotics is problematic because in recent years strains of plague bacteria have emerged that are resistant to one or more of the antibiotics traditionally employed to treat patients. Such resistance has been found to be encoded on transmissible plasmids (see, e.g., Galimand et al., N. Eng. J. Med., 337(10): 677-680 (1997); Dennis and Hughes, (1997), op. cit.).

The prospect of infection by inhalation or ingestion, combined with the viability of Y. pestis cells in the environment, make this species a potential bioterrorism threat. Y. pestis has been recognized as a category A agent of bioterrorism by the CDC. Intentional dissemination of plague would most probably occur via an aerosol of Y. pestis, a mechanism that has been shown to produce pneumonic plague in nonhuman primates (Inglesby, T. V., et al., JAMA, 283: 2281-2290 (2000)). WHO estimates that the dissemination of 50 kg of aerosolized Y. pestis over a population of 5 million would result in 150,000 infections and 36,000 deaths (Tjaden, J. A., et al., Postgrad. Med., 112: 57-60, 63-4, 67-70 (2002)).

The “Working Group on Civil Biodefense” concludes that an aerosolized plague weapon could cause signs consistent with severe pneumonia 1 to 6 days after exposure. Rapid evolution of disease would occur in the 2 to 4 days after symptom onset and would lead to septic shock with high mortality without early treatment (Inglesby, T. V., et al., (2000) op. cit.). Early treatment and prophylaxis with streptomycin or gentamicin or the tetracycline or fluoroquinolone classes of antimicrobials is recommended (Inglesby, T. V., et al. (2000), op. cit.). Delaying therapy until confirmatory testing is performed would greatly decrease survival, and no vaccine for Y. pestis has been approved for use in the US.

The potential for continued emergence and dissemination of resistant Y. pestis strains poses a global threat to public health as well as to biodefense. Since there is a significant risk of natural or intentional transfer of resistance to Y. pestis, and delays in selecting appropriate therapy are predicted to be very costly, new therapeutic agents that are not subject to existing resistance mechanisms and are capable of delaying progression of the disease will be crucial additions to the public and biodefense arsenal.

Novel therapeutics that target virulence factors offer the potential of providing those benefits.

Y. pestis is a Gram-negative facultative intracellular organism. Although the vast preponderance of bacterial cells are extracellular during infection, their ability to persist within phagocytic macrophages may contribute to virulence (Aleksic, S., et al. (ed.), Yersinia and other Enterobacteriaceae, 7th ed., ASM Press, Washington, D.C. (2003)).

In general, Y. pestis does not appear to kill its host by producing a potent toxin but by overcoming host resistance, generating massive growth and eventual septicemia (Cornelis, G. R., Proc. Natl. Acad. Sci. USA, 97: 8778-83 (2000); Sebbane, F., et al., Proc. Natl. Acad. Sci. USA, 103: 5526-30 (2006); Sodeinde, O. A., et al., Science, 258: 1004-7 (1992)). For example, in pneumonic plague, death may occur from pulmonary edema due to massive growth of Y. pestis in the lung before the development of septicemia (Lathem, W. W., et al., Science, 315: 509-13 (2007)).

In order to accomplish this rapid outgrowth in the host, Y. pestis employs multiple non-redundant virulence factors to evade the innate immune system and the ensuing proinflammatory response. Specifically, Y. pestis cells produce the following factors that enhance virulence: (a) an altered LPS, which is not recognized by toll-like receptor 4 (TLR4) (Montminy, S. W., et al., Nat. Immunol., 7: 1066-73 (2006)), (b) a membrane-embedded surface plasminogen activator (Pla), which facilitates dissemination by aiding suppression of local inflammation (Sodeinde, O. A., et al., (1992) op. cit.), and (c) a type three secretion system (T3SS) that actively suppresses inflammatory responses by injection of cellular toxins (Cornelis, G. R., (2000), op. cit.); Cornelis, G. R., Int. J. Med. Microbiol., 291: 455-62 (2002)). Specialized systems for the acquisition of iron are also required for full virulence (Bearden, S. W., et al., Mol. Microbiol., 32: 403-14 (1999); Bearden, S. W., et al., J. Bacteriol., 180: 1135-47 (1998)). Mechanistically, these various virulence factors are “non-redundant” because loss of each one individually has a significant effect on virulence despite the presence of the others. The latter three virulence factors, Pla, T3SS, and iron acquisition systems, could be susceptible to small-molecule inhibitors, and indeed efforts have been described to develop inhibitors for both T3SS and Pla (Agarkov, A., et al., Bioorg. Med. Chem. Lett., 18: 427-31 (2008); Kauppi, A. M., et al., Adv. Exp. Med. Biol., 529: 97-100 (2003); Pan, N. J., et al., Antimicrob. Agents Chemother., 53(2): 385-392 (2009)).

Only one screening project for Pla inhibitors has been described to date, and progress was confined to the development of fluorogenic peptide substrates to be used in high-throughput screening (Agarkov, A., et al., (2008), op. cit.).

Clearly, needs remain for new, potent inhibitors that target virulence factors against Y. pestis and other bacterial infections. Inhibitors that could be used during natural outbreaks or bio-terrorist attacks, and that could be used either prophylactically to treat a potentially exposed population or therapeutically after exposure or infection, administered alone or in combination with antibiotic therapy, would be especially desirable.

### SUMMARY OF THE INVENTION

The invention addresses the above needs by providing new inhibitor compounds of bacterial omptin proteases, specifically Yersinia pestis plasminogen activator (Pla), of different chemotypes. To identify Yersinia pestis plasminogen activator (Pla) entry inhibitor compounds described herein, a high throughput screen (HTS) assay was developed utilizing recombinant E. coli expressing Y. pestis Pla (with the 52251 chromogenic plasmin substrate) in the presence of plasminogen to identify putative entry inhibitors of Yersinia pestis plasminogen activator (Pla) and other bacterial omptin proteases. Libraries of thousands of discrete small molecule organic compounds and purified natural products were screened using this assay. The Yersinia pestis plasminogen activator (Pla) inhibitor compounds (“hits”) from the high throughput primary screen were then qualified through a series of secondary assays, including screens against tissue plasminogen activator (tPA), urokinase type plasminogen activator (uPA), human aspartyl proteases cathepsin D and E, and HIV-1 protease, as a counter screen to eliminate non-specific inhibitors, and cytotoxicity testing.

Accordingly, a Y. pestis plasminogen activator (Pla) inhibitor compound described herein inhibits the conversion of plasminogen to plasmin by plasminogen activator (Pla). Preferred Y. pestis Pla inhibitor compounds described herein inhibit and/or reduce dissemination of the bacterium in the infected host.

In preferred embodiments, a Y. pestis Pla inhibitor compound according to the present invention also inhibits activity of other bacterial omptin proteases, e.g., bacterial omptin proteases from Enterobacter cloacae, Escherichia coli, Escherichia coli (EPEC), Klebsiella oxytoca, Klebsiella pneumoniae, Salmonella ssp., and Shigella ssp.

The present invention provides isolated Y. pestis plasminogen activator inhibitor compounds of Formula (I):

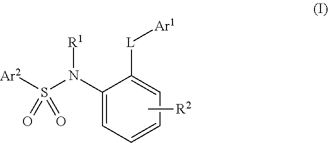

wherein:

L is a linker that is a direct bond or one of the following:

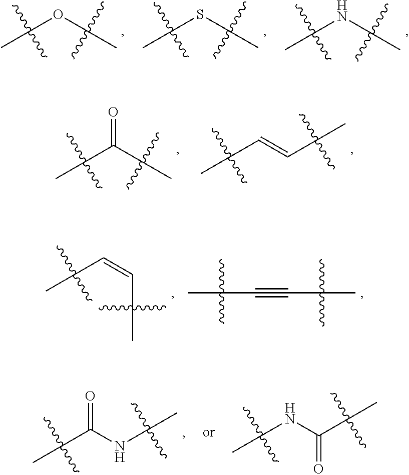

Ar¹ is a monovalent aryl or heteroaryl, cycloalkyl or heterocycloalkyl moiety which may be unsubstituted or substituted by up to 5 substituents selected from the group consisting of: halo, amino, amidino, guanidino, alkyl, haloalkyl, alkenyl, alkynyl, cycloalkyl, heterocycloalkyl, aryl, heteroaryl, hydroxy, alkoxy, aryloxy, heteroaryloxy, acyl, alkoxycarbonyl, aryloxycarbonyl, amino, substituted amino, acylamino, amido, sulfonamido, mercapto, alkylthio, arylthio, hydroxamate, thioacyl, alkylsulfonyl, or aminosulfonyl;

Ar² is a monovalent aryl or heteroaryl, moiety which may be unsubstituted or substituted by up to 5 substituents selected from the group consisting of: halo, amino, amidino, guanidino, alkyl, haloalkyl, alkenyl, alkynyl, cycloalkyl, heterocycloalkyl, aryl, heteroaryl, hydroxy, alkoxy, aryloxy, heteroaryloxy, acyl, carboxy, alkoxycarbonyl, aryloxycarbonyl, amino, substituted amino, acylamino, amido, sulfonamido, mercapto, alkylthio, arylthio, hydroxamate, thioacyl, alkylsulfonyl, or aminosulfonyl;

R¹ is a hydrogen or a monovalent alkyl, haloalkyl, alkenyl, alkynyl, cycloalkyl, heterocycloalkyl, or acyl moiety; and

R² represents a single or multiple substituents selected from the group consisting of: halo, amino, amidino, guanidino, alkyl, haloalkyl, alkenyl, alkynyl, cycloalkyl, heterocycloalkyl, aryl, heteroaryl, hydroxy, alkoxy, aryloxy, heteroaryloxy, acyl, carboxy, alkoxycarbonyl, aryloxycarbonyl, amino, substituted amino, acylamino, amido, sulfonamido, mercapto, alkylthio, arylthio, hydroxamate, thioacyl, alkylsulfonyl, or aminosulfonyl, located at the 3-, 4-, 5-, or 6-position of the phenyl ring;

and pharmaceutically acceptable salts thereof.

In preferred embodiments, the present invention provides isolated Y. pestis plasminogen activator inhibitor compounds of the formulae:

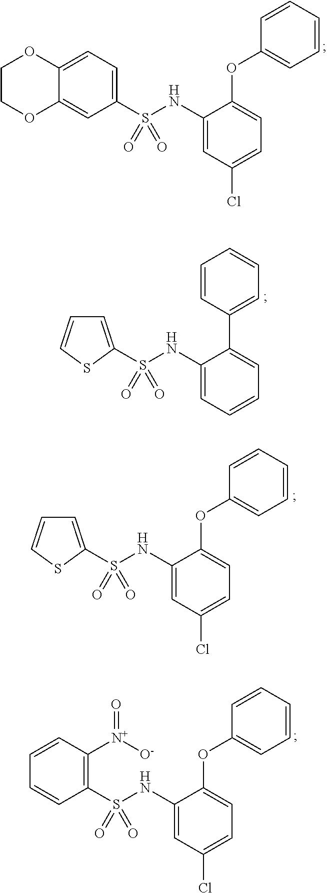

and pharmaceutically acceptable salts thereof.

The present invention further provides isolated Y. pestis plasminogen activator inhibitor compounds of the formula:

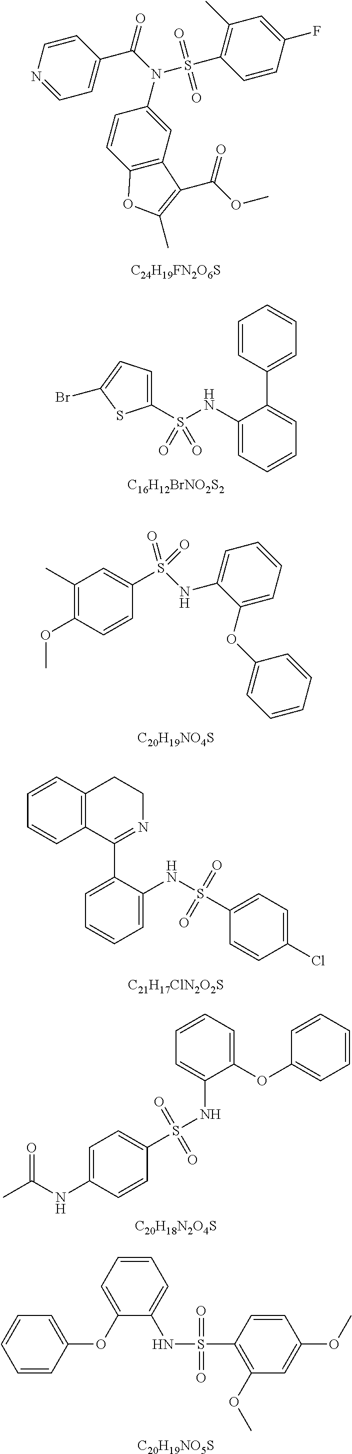

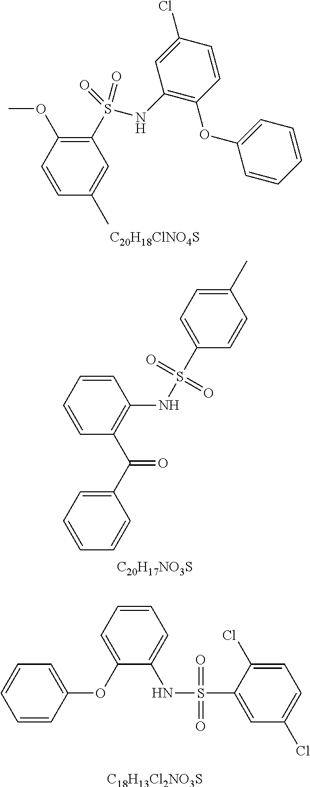

and pharmaceutically acceptable salts thereof.

The foregoing compounds were identified by assays showing specific inhibition of Yersinia pestis plasminogen activator (Pla) and other bacterial omptin proteases.

Y. pestis plasminogen activator (Pla) inhibitory properties discovered for the compounds of the invention are set forth in Tables 2-3, and FIG. 6 infra. Inhibitor compounds were identified in the assays described herein as inhibiting plasminogen activator (as measured by the reduction in the cleavage chromogenic plasmin substrate vs. control) by at least 50% at a concentration of 50 μM using a recombinant E. coli expressing Y. pestis Pla (with the S2251 chromogenic plasmin substrate) in the presence of plasminogen. Compounds inhibiting Y. pestis plasminogen activator (Pla) by less than 50% or with a CC₅₀ greater than 50 μM are not generally useful as Y. pestis plasminogen activator inhibitors in the compositions and methods of treatment (medical uses) described herein. For alternative uses such as on surfaces, e.g., as a disinfectant, compounds of less potency and greater cytotoxicity may be advantageously employed.

In a particularly preferred embodiment, a Y. pestis Pla inhibitor compound useful in the compositions and methods described herein has an IC₅₀ of less than 25 μM as measured in recombinant E. coli expressing Y. pestis Pla (with the S2251 chromogenic plasmin substrate) in the presence of plasminogen assay (described herein or comparable assay) and also has a relatively low cytotoxicity toward human cells, such as a CC₅₀ value of greater than or equal to 50 μM (CC₅₀>50 μM) as measured in a standard cytotoxicity assay as described herein or as employed in the pharmaceutical field for antibacterial agents. Such standard cytotoxicity assays may employ any mammalian cell typically employed in cytotoxicity assays for antibiotics, including but not limited to, Chinese hamster ovary (CHO) cells, Vero (African green monkey kidney) cells, HeLa cells, Hep-G2 (human hepatocellular carcinoma) cells, human embryonic kidney (HEK) 293 cells, 293T cells, 293FT cells (Invitrogen), BHK (newborn hamster kidney) cells, COS cells, and the like.

The Y. pestis plasminogen activator inhibitor compounds described herein are useful as antibacterial agents and may be used to treat bacterial infection, either prophylactically when administered to an individual or a potentially exposed population or therapeutically during the post-infection period. Accordingly, an individual infected with a bacterium, particularly, Yersina pestis, or exposed to Y. pestis infection, may be treated by administering to the individual in need an effective amount of a compound according to the invention, e.g., administering one or more of the compounds of Formula (I):

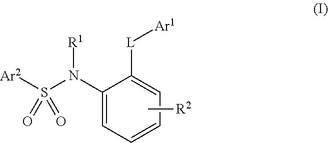

wherein:

L is a linker that is a direct bond or one of the following:

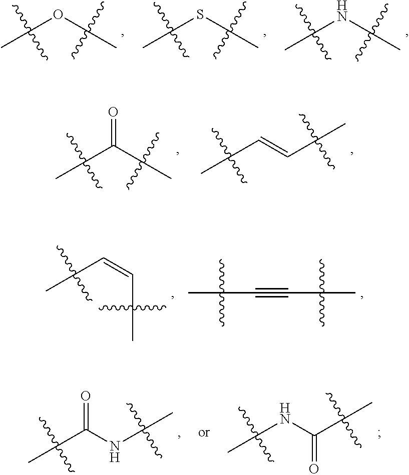

Ar¹ is a monovalent aryl or heteroaryl, cycloalkyl or heterocycloalkyl moiety which may be unsubstituted or substituted by up to 5 substituents from the groups of halo, amino, amidino, guanidino, alkyl, haloalkyl, alkenyl, alkynyl, cycloalkyl, heterocycloalkyl, aryl, heteroaryl, hydroxy, alkoxy, aryloxy, heteroaryloxy, acyl, alkoxycarbonyl, aryloxycarbonyl, amino, substituted amino, acylamino, amido, sulfonamido, mercapto, alkylthio, arylthio, hydroxamate, thioacyl, alkylsulfonyl, or aminosulfonyl;

Ar² is a monovalent aryl or heteroaryl, moiety which may be unsubstituted or substituted by up to 5 substituents from the groups of halo, amino, amidino, guanidino, alkyl, haloalkyl, alkenyl, alkynyl, cycloalkyl, heterocycloalkyl, aryl, heteroaryl, hydroxy, alkoxy, aryloxy, heteroaryloxy, acyl, carboxy, alkoxycarbonyl, aryloxycarbonyl, amino, substituted amino, acylamino, amido, sulfonamido, mercapto, alkylthio, arylthio, hydroxamate, thioacyl, alkylsulfonyl, or aminosulfonyl;

R¹ is a hydrogen or a monovalent alkyl, haloalkyl, alkenyl, alkynyl, cycloalkyl, heterocycloalkyl, or acyl moiety; and

R² represents a single or multiple substituents from the list of: halo, amino, amidino, guanidino, alkyl, haloalkyl, alkenyl, alkynyl, cycloalkyl, heterocycloalkyl, aryl, heteroaryl, hydroxy, alkoxy, aryloxy, heteroaryloxy, acyl, carboxy, alkoxycarbonyl, aryloxycarbonyl, amino, substituted amino, acylamino, amido, sulfonamido, mercapto, alkylthio, arylthio, hydroxamate, thioacyl, alkylsulfonyl, or aminosulfonyl, located at the 3-, 4-, 5-, or 6-position of the phenyl ring;

and pharmaceutically acceptable salts thereof.

In preferred embodiments, an individual infected with a bacterium, particularly, Yersina pestis, or exposed to Y. pestis infection, may be treated by administering to the individual in need an effective amount of a compound according to the invention, e.g., administering one or more of the following compounds or pharmaceutically acceptable salts thereof:

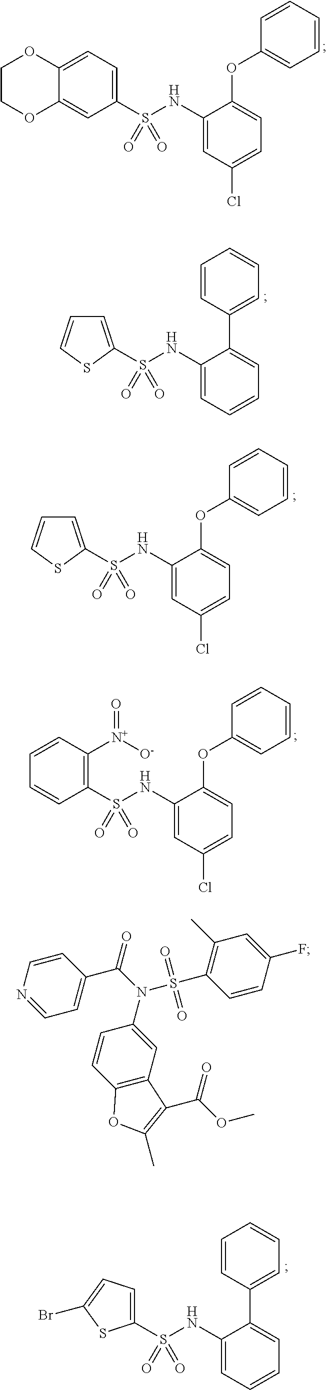

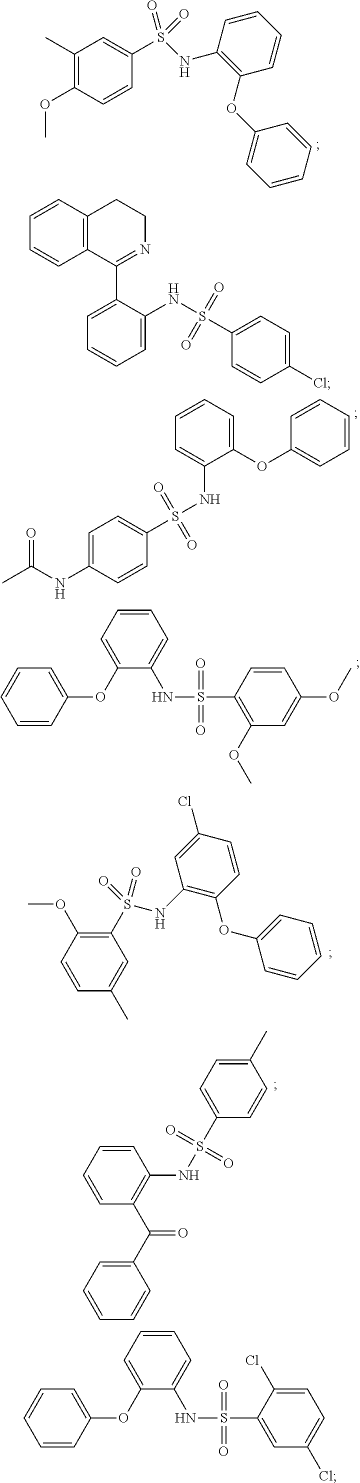

and pharmaceutically acceptable salts thereof.

Use of one or more or a combination of the above compounds to inhibit Y. pestis plasminogen activator is contemplated herein. Especially, use of one or more or a combination of the above compounds to treat plague is contemplated herein. In particular, use of one or more or a combination of the above compounds for the treatment of infection of bubonic (infection of lymph nodes), pneumonic (infection of lungs), or blood (septicemic) forms of the disease, is advantageously carried out by following the teachings herein.

Use of one or more or a combination of the above compounds to prepare a medicament for treating Y. pestis infection is contemplated herein.

The present invention also provides pharmaceutical compositions containing one or more of the Y. pestis plasminogen activator inhibitor compounds disclosed herein and a pharmaceutically acceptable carrier or excipient. The use of one or more of the Y. pestis plasminogen activator inhibitor compounds in the preparation of a medicament for combating Y. pestis infection is disclosed.

In yet another embodiment, a composition comprising a Y. pestis plasminogen activator inhibitor or a combination of Y. pestis plasminogen activator inhibitors described herein may also comprise a second agent (second active ingredient, second active agent) that possesses a desired therapeutic or prophylactic activity other than that of Y. pestis plasminogen activator inhibition. Such a second active agent includes, but is not limited to, an antibiotic, an antibody, an antiviral agent, an anticancer agent, an analgesic (e.g., a non-steroidal anti-inflammatory drug (NSAID), acetaminophen, an opioid, a COX-2 inhibitor), an immunostimulatory agent (e.g., a cytokine), a hormone (natural or synthetic), a central nervous system (CNS) stimulant, an antiemetic agent, an anti-histamine, an erythropoietin, a complement stimulating agent, a sedative, a muscle relaxant agent, an anesthetic agent, an anticonvulsive agent, an antidepressant, an antipsychotic agent, a type three secretion system (T3SS) inhibitor, and combinations thereof.

Compositions comprising a Y. pestis Pla inhibitor described herein may be formulated for administration to an individual (human or other animal) by any of a variety of routes including, but not limited to, intravenous, intramuscular, subcutaneous, intra-arterial, parenteral, intraperitoneal, sublingual (under the tongue), buccal (cheek), oral (for swallowing), topical (epidermis), transdermal (absorption through skin and lower dermal layers to underlying vasculature), nasal (nasal mucosa), intrapulmonary (lungs), intrauterine, vaginal, intracervical, rectal, intraretinal, intraspinal, intrasynovial, intrathoracic, intrarenal, nasojejunal, and intraduodenal.

### BRIEF DESCRIPTION OF THE DRAWINGS

FIG. 1 is a structural model of Y. pestis Pla based on the structure of E. coli OmpT (PDB 1178), the prototype member of the omptin family, as a template. Pla (3-barrel structure reveals the basic arrangement of the protein in the membrane with the active site residing in exposed loops outside the cell. The position of the outer membrane bilayer is indicated. Conserved catalytic residues D206, and H208 in the solvent exposed loop regions (L1-L5) are identified. The putative LPS-binding amino acids are also indicated: R171 and R138 and residues Y134 and E136 are predicted to bind lipid A.

FIG. 2 is a graph showing the results of the development and validation of the high-throughput screen assay for inhibitors of Y. pestis Pla. In the simplest configuration, Pla (as membranes or intact bacteria) is mixed with D-Val-Leu-Lys-p-nitroanalide (trade name S2251), which is a chromogenic plasmin substrate, and the reaction is started by addition of plasminogen. In this coupled assay, color development due to the substrate cleavage by plasmin created in the reaction follows a parabolic trajectory because the amount of plasmin is continuously increasing. Assays were carried out in 100 μL volume in 384-well microplates using 4 μL of E. coli BL21(pBSpla) cells and reagent concentrations as described herein. Panel A: Time course of the reaction, following 16 wells each of the complete reaction (negative control), the complete reaction with 16 mM NH₂-Lys-Val inhibitor (inhibitor control), and the complete reaction except for Glu-PLG (positive control). A₄₀₅ was measured over four 30-min periods and plotted for each well. Panel B: Endpoint values at 120 min for a complete 384-well microplate containing positive (160 wells) and negative (160 wells) controls and 16 mM NH₂-Lys-Val inhibitor (64 wells). The Z′ value reached 0.6 by 120 min.

FIG. 3 shows histopathologic slides exemplifying the lack of accumulation of inflammatory cells, particularly neutrophils, in mice infected intravenously with 1,000 Y. pestis. Mice are infected intravenously with 1,000 Y. pestis, and at two days post-infection are sacrificed and their livers examined histologically. At the time of infection, some bacteria are deposited in the liver and the extent of inflammatory cell infiltration at the sites of colonization is readily seen in hematoxylin-eosin (H+E) stained sections. When wild-type bacteria are used, masses of bacteria are seen packed in liver sinusoids (see, FIG. 3A, arrow) but virtually no inflammatory cells are present, a remarkable demonstration of the ability of Y. pestis to suppress and evade innate immune defenses. In contrast, when Pla-deficient bacteria are used, few free bacteria are visible in the liver. Instead, microabscesses consisting of masses of inflammatory cells surrounding the bacteria are observed (see, FIG. 3B, asterisk).

FIG. 4 is a chart showing the results of the survey of Pla-like activity in selected clinical isolates of 41 species of Enterbacteriacea using plasminogen as substrate. Each dot represents the Pla activity from a specific bacterial strain of the species indicated. Results show that this activity is widespread and present in species not previously known to have omptin members, including Enterobacter cloacae and Klebsiella pneumoniae. A clear bimodal distribution of activity for some species is evident. It is possible that these high-activity subsets are associated with more severe disease.

FIG. 5 is a workflow diagram illustrating the selection process for Yersinia pestis plasminogen activator (Pla) inhibitor compounds according to the invention. From an initial composite collection of 109,265 small molecule compounds and natural products at 50 μM concentration, compounds showing greater than 50% inhibition of Pla were selected and retested in 4 confirmation assay plates. Compounds showing greater than 50% inhibition of Pla with a z-score greater than 3 in at least 3 of 4 replicated assays were further selected and IC₅₀ values were calculated. Compounds having an IC₅₀≦25 μM were further tested for cytotoxicity. Compounds proving to have low cytotoxicity (CC₅₀ greater than 50 μM) were selected for further study.

FIG. 6 is a graph showing the potency (IC₅₀) and cytotoxicity (CC₅₀) of confirmed inhibitor Compound 3.

### DETAILED DESCRIPTION OF THE INVENTION

The invention provides organic compounds that inhibit bacterial omptin proteases, specifically Yersinia pestis plasminogen activator (Pla).

In order that the invention may be more clearly understood, the following abbreviations and terms are used as defined below.

Abbreviations for various substituents (side groups, radicals) of organic molecules are those commonly used in organic chemistry. Such abbreviations may include “shorthand” forms of such substituents. For example, “Me” and “Et” are abbreviations used to indicate methyl (CH₃—) and ethyl (CH₃CH₂—) groups, respectively; and “OMe” and “OEt” indicate methoxy (CH₃O—) and ethoxy (CH₃CH₂O—), respectively. Hydrogen atoms are not always shown in organic molecular structures or may be only selectively shown in some structures, as the presence and location of hydrogen atoms in organic molecular structures are understood and known by persons skilled in the art. Likewise, carbon atoms are not always specifically abbreviated with “C”, as the presence and location of carbon atoms, e.g., between or at the end of bonds, in structural diagrams are known and understood by persons skilled in the art. Minutes are commonly abbreviated as “min”; hours are commonly abbreviated as “hr” or “h”.

A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step. It is also understood that an element or step “selected from the group consisting of” refers to one or more of the elements or steps in the list that follows, including combinations of any two or more of the listed elements or steps.

In the context of therapeutic use of the Y. pestis plasminogen activator inhibitor compounds described herein, the terms “treatment”, “to treat”, or “treating” will refer to any use of the Y. pestis plasminogen activator inhibitor compounds calculated or intended to arrest, inhibit, prevent or reduce the infection of a host cell with a Y. pestis by inhibiting the activity of virulence factor Pla. Thus, treating an individual may be carried out after any diagnosis indicating possible Y. pestis infection, i.e., whether an infection by Y. pestis has been confirmed or whether the possibility of infection is only suspected, for example, after an individual's exposure to Y. pestis or to another individual infected by Y. pestis. It is also recognized that because the inhibitors of the present invention affect the dissemination of the bacteria in the host organism, the inhibitors disclosed herein will also be useful for delaying the progression of the infection in those exposed but whose infection or development of disease has not been confirmed or diagnosed. Also, because the compounds of the present invention inhibit Pla, it will be understood that elimination of the bacterial infection will be accomplished by the host's own immune system or immune effector cells. Thus, it is contemplated that the compounds of the present invention will often be routinely combined with other active ingredients such as antibiotics, antibodies, antiviral agents, anticancer agents, analgesics (e.g., a non-steroidal anti-inflammatory drug (NSAID), acetaminophen, opioids, COX-2 inhibitors), immunostimulatory agents (e.g., cytokines or a synthetic immunostimulatory organic molecules), hormones (natural, synthetic, or semi-synthetic), central nervous system (CNS) stimulants, antiemetic agents, anti-histamines, erythropoietin, agents that activate complement, sedatives, muscle relaxants, anesthetic agents, anticonvulsive agents, antidepressants, antipsychotic agents, a type three secretion system (T3SS) inhibitor, and combinations thereof.

The terms “halo” or “halogen” as used herein refer to fluorine, chlorine, bromine, or iodine.

The term “alkyl” is intended to include a straight or branched chain monovalent or divalent radical of saturated carbon atoms and hydrogen atoms, such as methyl (Me), ethyl (Et), propyl (Pr), isopropyl (iPr), butyl (Bu), isobutyl (iBu), sec-butyl (sBu), ten-butyl (tBu), and the like, which may be unsubstituted, or substituted by one or more suitable substituents found herein.

The term “haloalkyl” is intended to mean an alkyl moiety that is substituted with one or more identical or different halogen atoms, e.g., —CH₂Cl, —CF₃, —CH₂CF₃, —CH₂CCl₃, and the like.

The term “alkenyl” is intended to mean a straight-chain, branched, or cyclic hydrocarbon radical having from between 2-8 carbon atoms and at least one double bond, e.g., ethenyl, 3-buten-1-yl, 3-hexen-1-yl, cyclopent-1-en-3-yl, and the like, which may be unsubstituted, or substituted by one or more suitable substituents found herein.

The term “alkynyl” as used herein refers to mean a straight-chain or branched hydrocarbon radical having from between 2-8 carbon atoms an at least one triple bond, e.g., ethynyl, 3-butyn-1-yl, 2-butyn-1-yl, 3-pentyn-1-yl, and the like, which may be unsubstituted, or substituted by one or more suitable substituents found herein.

The term “cycloalkyl” is intended to mean a non-aromatic monovalent, monocyclic or polycyclic radical having from between 3-12 carbon atoms, each of which may be saturated or unsaturated, e.g., cyclopentyl, cyclohexyl, decalinyl, and the like, unsubstituted, or substituted by one or more of the suitable substituents found herein, and to which may be fused one or more aryl groups, heteroaryl groups, or heterocycloalkyl groups, which themselves may be unsubstituted or substituted by one or more suitable substituents found herein.

The term “heterocycloalkyl” is intended to mean a non-aromatic monovalent, monocyclic or polycyclic radical having from between 2-12 carbon atoms, and between 1-5 heteroatoms selected from nitrogen, oxygen, or sulfur, each of which may be saturated or unsaturated, e.g., pyrrolodinyl, tetrahydropyranyl, morpholinyl, piperazinyl, oxiranyl, and the like, unsubstituted, or substituted by one or more of the suitable substituents found herein, and to which may be fused one or more aryl groups, heteroaryl groups, or heterocycloalkyl groups, which themselves may be unsubstituted or substituted by one or more suitable substituents found herein.

As used herein, the term “aryl” is intended to mean an aromatic monovalent, monocyclic or polycyclic radical comprising between 6 and 18 carbon ring members, e.g., phenyl, biphenyl, naphthyl, phenanthryl, and the like, which may be substituted by one or more of the suitable substituents found herein, and to which may be fused one or more heteroaryl groups or heterocycloalkyl groups, which themselves may be unsubstituted or substituted by one or more suitable substituents found herein.

The term “heteroaryl” is intended to mean an aromatic monovalent monocyclic or polycyclic radical comprising between 2 and 18 carbon ring members and at least 1 heteroatom selected from nitrogen, oxygen, or sulfur, e.g., pyridyl, pyrazinyl, pyridizinyl, pyrimidinyl, furanyl, thienyl, triazolyl, quinolinyl, imidazolinyl, benzimidazolinyl, indolyl, and the like, which may be substituted by one or more of the suitable substituents found herein, and to which may be fused one or more aryl, heteroaryl groups or heterocycloalkyl groups, which themselves may be unsubstituted or substituted by one or more suitable substituents found herein.

The term “hydroxy” is intended to mean the radical —OH.

The term “alkoxy” is intended to mean the radical —OR where R is an alkyl or cycloalkyl group.

As used herein, the term “aryloxy” is intended to mean the radical —OAr where Ar is an aryl group.

The term “heteroaryloxy” refers to radical —O(HAr) where HAr is a heteroaryl group.

The term “acyl” is intended to mean a —C(O)R radical where R is alkyl, alkenyl, alkynyl, cycloalkyl, aryl, heteroaryl, or heterocycloalkyl, e.g. acetyl, benzoyl, and the like.

The term “carboxy” is intended to mean the radical —C(O)OH.

The term “alkoxycarbonyl” is intended to mean a —C(O)OR radical where R is alkyl, alkenyl, alkynyl, or cycloalkyl.

The term “aryloxycarbonyl” is intended to mean a —C(O)OR radical where R is aryl or heteroaryl.

As used herein, the term “amino” refers to the radical —NH₂.

The term “substituted amino” is intended to mean the radical —NRR′ where R, and R′ are, independently, hydrogen, alkyl, alkenyl, alkynyl, cycloalkyl, aryl, or heteroaryl, or heterocycloalkyl.

The term “acylamino” is intended to mean the radical —NHC(O)R, where R is alkyl, alkenyl, alkynyl, cycloalkyl, aryl, heteroaryl, or heterocycloalkyl, e.g. acetyl, benzoyl, and the like, e.g., acetylamino, benzoylamino, and the like.

The term “amido” in intended to mean the radical —C(O)NRR′ where R and R′ are, independently, hydrogen, alkyl, alkenyl, alkynyl, cycloalkyl, aryl, or heteroaryl, or heterocycloalkyl.

The term “sulfonylamino” is intended to mean the radical —NHSO₂R where R is alkyl, alkenyl, alkynyl, cycloalkyl, aryl, heteroaryl, or heterocycloalkyl.

The term “amidino” is intended to mean the radical —C(NR)NR′R″, where R, R′, and R″ are, independently, hydrogen, alkyl, alkenyl, alkynyl, cycloalkyl, aryl, or heteroaryl, and wherein R, R′, and R″ may form heterocycloalkyl rings, e.g. carboxamido, imidazolinyl, tetrahydropyrimidinyl.

The term “guanidino” is intended to mean the radical —NHC(NR)NR′R″, where R, R′, and R″ are, independently, hydrogen, alkyl, alkenyl, alkynyl, cycloalkyl, aryl, or heteroaryl, and wherein R, R′, and R″ may form heterocycloalkyl rings.

The term “mercapto” as used herein refers to the radical —SH.

The term “alkylthio” is intended to mean the radical —SR where R is an alkyl or cycloalkyl group.

The term “arylthio” is intended to mean the radical —SAr where Ar is an aryl group.

The term “hydroxamate” is intended to mean the radical —C(O)NHOR where R is an alkyl or cycloalkyl group.

The term “thioacyl” is intended to mean a —C(S)R radical where R is alkyl, alkenyl, alkynyl, cycloalkyl, aryl, heteroaryl, or heterocycloalkyl.

The term “alkylsulfonyl” is intended to mean the radical —SO₂R where R is alkyl, alkenyl, alkynyl, cycloalkyl, aryl, heteroaryl, or heterocycloalkyl.

The term “aminosulfonyl” is intended to mean the radical —SO₂NRR′ where R and R′ are, independently, hydrogen, alkyl, alkenyl, alkynyl, cycloalkyl, aryl, or heteroaryl, or heterocycloalkyl.

The meaning of other terms will be understood by the context as understood by the skilled practitioner in the art, including the fields of organic chemistry, pharmacology, and bacteriology.

The invention provides specific organic compounds that inhibit bacterial omptin proteases, particularly Y. pestis plasminogen activator (Pla). Putative inhibitors of Y. pestis Pla (“hits”) were initially identified by screening collections of organic molecules using a high throughput screen (HTS) assay utilizing recombinant E. coli expressing Y. pestis Pla (with the S2251 chromogenic plasmin substrate) in the presence of plasminogen. Compounds showing greater than 50% inhibition of Pla at a 50 μM concentration were designated as a “hit”. Most (e.g., greater than 80%) of the initial hits were subsequently eliminated by confirmation retesting and a counter assay (to eliminate non-specific inhibitors) measuring inhibitory effect against human tissue plasminogen activator (tPA), human urokinase type plasminogen activator (uPA), human aspartyl proteases cathepsin D and E, and HIV-1 protease. Non-specific inhibitor compounds were discarded.

A Y. pestis plasminogen activator inhibitor compound useful in the compositions and methods of the invention has a structure as shown in Formula (I) or in Tables 2 or 3 infra. The compounds preferably have a 50% inhibitory concentration (IC₅₀) less than 100 μM, preferably less than 25 μM, most preferably less than 10 μM, as measured in a suitable assay, such as a high throughput screen (HTS) assay utilizing recombinant E. coli expressing Y. pestis Pla (with the S2251 chromogenic plasmin substrate) in the presence of plasminogen as described in the examples, infra. Compounds with IC₅₀ greater than 100 μM are not generally useful as therapeutic inhibitors in the compositions and methods described herein for administration to humans and other animals.

A Y. pestis plasminogen activator inhibitor compound that is particularly useful in the compositions and methods described herein has an IC₅₀ of less than 100 μM as measured in a suitable assay, such as a high throughput screen (HTS) assay utilizing recombinant E. coli expressing Y. pestis Pla (with the S2251 chromogenic plasmin substrate) in the presence of plasminogen (or comparable assay) and also has a relatively low cytotoxicity toward mammalian cells, such as a CC₅₀ value of greater than or equal to 50 μM as measured in a standard cytotoxicity assay as described herein or as employed in the pharmaceutical field for antibacterial agents. Such standard cytotoxicity assays may employ Chinese hamster ovary (CHO) cells, HeLa cells, Hep-G2 cells, human embryonic kidney (HEK) 293 cells, 293T cells, 293FT cells, BHK cells, COS cells or other suitable mammalian cell lines known in the art.

Preferred Y. pestis plasminogen activator inhibitor compounds described herein include compounds of Formula (I), compounds as depicted in Tables 2 and 3 infra, and combinations thereof.

The Y. pestis plasminogen activator inhibitor compounds described herein are organic compounds that can be either synthesized or ordered from suppliers such as Maybridge (Cornwall, UK), Microsource Discovery Systems, Inc. (Gaylordsville, Conn., USA), Chemical Diversity Labs (San Diego, Calif., USA), ChemBridge Corp. (DIVERSet™; San Diego, Calif., USA), and TimTec, Inc. (Newark, Del., USA). The Y. pestis plasminogen activator inhibitor compounds described herein may also be synthesized using established chemistries, and suitable synthesis schemes for the compounds include the following:

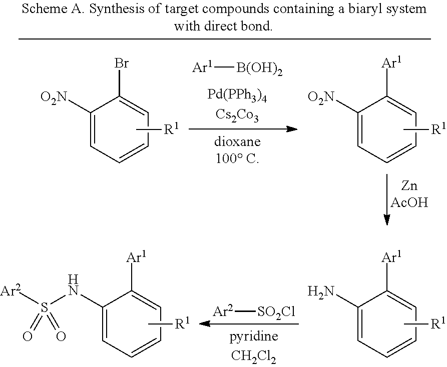

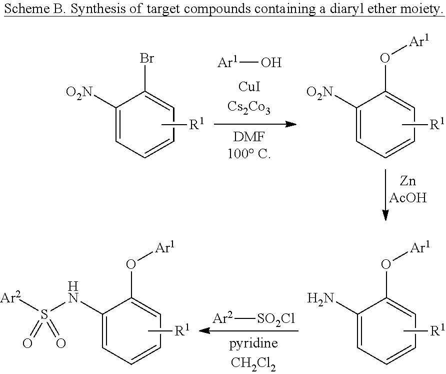

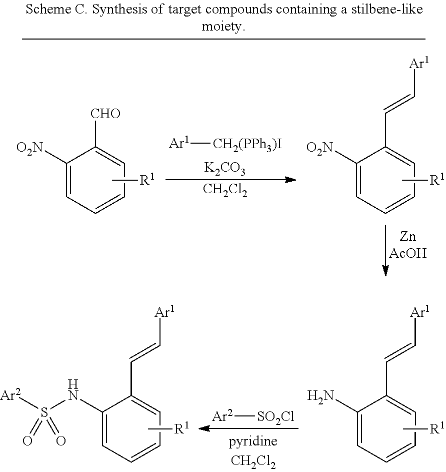

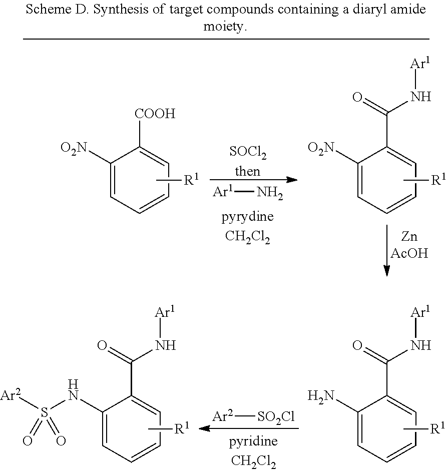

Unless otherwise indicated, it is understood that description of the use of a Y. pestis plasminogen activator inhibitor compound in a composition or method also encompasses embodiments wherein a combination of two or more Y. pestis plasminogen activator inhibitor compounds are employed as active ingredients providing Y. pestis plasminogen activator inhibitory activity in a composition or method of the invention.

Pharmaceutical compositions according to the invention comprise an isolated Y. pestis plasminogen activator inhibitor compound as described herein, or a pharmaceutically acceptable salt thereof, as the active ingredient and a pharmaceutically acceptable carrier (or “vehicle”), which may be a liquid, solid, or semi-solid compound. By “pharmaceutically acceptable” is meant that a compound or composition is not biologically, chemically, or in any other way, incompatible with body chemistry and metabolism and also does not adversely affect the Y. pestis plasminogen activator inhibitor or any other component that may be present in a composition in such a way that would compromise the desired therapeutic and/or preventative benefit to a patient. Pharmaceutically acceptable carriers useful in the invention include those that are known in the art of preparation of pharmaceutical compositions and include, without limitation, water, physiological pH buffers, physiologically compatible salt solutions (e.g., phosphate buffered saline), and isotonic solutions. Pharmaceutical compositions of the invention may also comprise one or more excipients, i.e., compounds or compositions that contribute or enhance a desirable property in a composition other than the active ingredient.

Various aspects of formulating pharmaceutical compositions, including examples of various excipients, dosages, dosage forms, modes of administration, and the like are known to those skilled in the art of pharmaceutical compositions and also available in standard pharmaceutical texts, such as Remington's Pharmaceutical Sciences, 18th edition, Alfonso R. Gennaro, ed. (Mack Publishing Co., Easton, Pa. 1990), Remington: The Science and Practice of Pharmacy, Volumes 1 & 2, 19th edition, Alfonso R. Gennaro, ed., (Mack Publishing Co., Easton, Pa. 1995), or other standard texts on preparation of pharmaceutical compositions.

Pharmaceutical compositions may be in any of a variety of dosage forms particularly suited for an intended mode of administration. Such dosage forms, include, but are not limited to, aqueous solutions, suspensions, syrups, elixirs, tablets, lozenges, pills, capsules, powders, films, suppositories, and powders, including inhalable formulations. Preferably, the pharmaceutical composition is in a unit dosage form suitable for single administration of a precise dosage, which may be a fraction or a multiple of a dose that is calculated to produce effective inhibition of Y. pestis plasminogen activator.

A composition comprising a Y. pestis plasminogen activator inhibitor compound (or combination of Y. pestis plasminogen activator inhibitors) described herein may optionally possess a second active ingredient (also referred to as “second agent”, “second active agent”) that provides one or more other desirable therapeutic or prophylactic activities other than Y. pestis plasminogen activator inhibitory activity. Suitable second agents useful in compositions of the invention include, but without limitation, an antibiotic, an antibody, an antiviral agent, an anticancer agent, an analgesic (e.g., a non-steroidal anti-inflammatory drug (NSAID), acetaminophen, an opioid, a COX-2 inhibitor), an immunostimulatory agent (e.g., a cytokine or a synthetic immunostimulatory organic molecule), a hormone (natural, synthetic, or semi-synthetic), a central nervous system (CNS) stimulant, an anti-emetic agent, an anti-histamine, an erythropoietin, a complement stimulating agent, a sedative, a muscle relaxant agent, an anesthetic agent, an anticonvulsive agent, an antidepressant, an antipsychotic agent, pluralities of such agents, a type three secretion system (T3SS) inhibitor, and combinations thereof.

Pharmaceutical compositions as described herein may be administered to humans and other animals in a manner similar to that used for other known therapeutic or prophylactic agents, and particularly as used for therapeutic antibiotics. The dosage to be administered to an individual and the mode of administration will depend on a variety of factors including age, weight, sex, condition of the patient, and genetic factors, and will ultimately be decided by an attending qualified healthcare provider.

Pharmaceutically acceptable salts of Y. pestis plasminogen activator inhibitor compounds described herein include those derived from pharmaceutically acceptable inorganic and organic acids and bases. Examples of suitable acids include hydrochloric, hydrobromic, sulfuric, nitric, perchloric, fumaric, maleic, malic, pamoic, phosphoric, glycolic, lactic, salicylic, succinic, toluene-p-sulfonic, tartaric, acetic, citric, methanesulfonic, formic, benzoic, malonic, naphthalene-2-sulfonic, tannic, carboxymethyl cellulose, polylactic, polyglycolic, and benzenesulfonic acids.

For solid compositions, conventional nontoxic solid carriers may be used including, but not limited to, mannitol, lactose, starch, magnesium stearate, sodium saccharin, talc, cellulose, glucose, sucrose, and magnesium carbonate.

Pharmaceutical compositions may be formulated for administration to a patient by any of a variety of parenteral and non-parenteral routes or modes. Such routes include, without limitation, intravenous, intramuscular, intra-articular, intraperitoneal, intracranial, paravertebral, periarticular, periostal, subcutaneous, intracutaneous, intrasynovial, intrasternal, intrathecal, intralesional, intratracheal, sublingual, pulmonary, topical, rectal, nasal, buccal, vaginal, or via an implanted reservoir. Implanted reservoirs may function by mechanical, osmotic, or other means. Generally and particularly when administration is via an intravenous, intra-arterial, or intramuscular route, a pharmaceutical composition may be given as a bolus, as two or more doses separated in time, or as a constant or non-linear flow infusion.

A pharmaceutical composition may be in the form of a sterile injectable preparation, e.g., as a sterile injectable aqueous solution or an oleaginous suspension. Such preparations may be formulated according to techniques known in the art using suitable dispersing or wetting agents (e.g., polyoxyethylene 20 sorbitan monooleate (also referred to as “polysorbate 80”); TWEEN® 80, ICI Americas, Inc., Bridgewater, N.J.) and suspending agents. Among the acceptable vehicles and solvents that may be employed for injectable formulations are mannitol, water, Ringer's solution, isotonic sodium chloride solution, and a 1,3-butanediol solution. In addition, sterile, fixed oils may be conventionally employed as a solvent or suspending medium. For this purpose, a bland fixed oil may be employed including synthetic mono- or diglycerides. Fatty acids, such as oleic acid and its glyceride derivatives are useful in the preparation of injectables, as are natural pharmaceutically-acceptable oils, including olive oil or castor oil, especially in their polyoxyethylated versions.

A Y. pestis plasminogen activator inhibitor described herein may be formulated in any of a variety of orally administrable dosage forms including, but not limited to, capsules, tablets, caplets, pills, films, aqueous solutions, oleaginous suspensions, syrups, or elixirs. In the case of tablets for oral use, carriers, which are commonly used include lactose and corn starch. Lubricating agents, such as magnesium stearate, are also typically added. For oral administration in a capsule form, useful diluents include lactose and dried cornstarch. Capsules, tablets, pills, films, lozenges, and caplets may be formulated for delayed or sustained release.

Tablets and other solid or semi-solid formulations may be prepared that rapidly disintegrate or dissolve in an individual's mouth. Such rapid disintegration or rapid dissolving formulations may eliminate or greatly reduce the use of exogenous water as a swallowing aid. Furthermore, rapid disintegration or rapid dissolve formulations are also particularly useful in treating individuals with swallowing difficulties. For such formulations, a small volume of saliva is usually sufficient to result in tablet disintegration in the oral cavity. The active ingredient (a Y. pestis plasminogen activator inhibitor described herein) can then be absorbed partially or entirely into the circulation from blood vessels underlying the oral mucosa (e.g., sublingual and/or buccal mucosa), or it can be swallowed as a solution to be absorbed from the gastrointestinal tract.

When aqueous suspensions are to be administered orally, whether for absorption by the oral mucosa or absorption via the gut (stomach and intestines), a composition comprising a Y. pestis plasminogen activator inhibitor may be advantageously combined with emulsifying and/or suspending agents. Such compositions may be in the form of a liquid, dissolvable film, dissolvable solid (e.g., lozenge), or semi-solid (chewable and digestible). If desired, such orally administrable compositions may also contain one or more other excipients, such as a sweetener, a flavoring agent, a taste-masking agent, a coloring agent, and combinations thereof.

The pharmaceutical compositions comprising a Y. pestis plasminogen activator inhibitor as described herein may also be formulated as suppositories for vaginal or rectal administration. Such compositions can be prepared by mixing a Y. pestis plasminogen activator inhibitor compound as described herein with a suitable, non-irritating excipient that is solid at room temperature but liquid at body temperature and, therefore, will melt in the appropriate body space to release the Y. pestis plasminogen activator inhibitor and any other desired component of the composition. Excipients that are particularly useful in such compositions include, but are not limited to, cocoa butter, beeswax, and polyethylene glycols.

Topical administration of a Y. pestis plasminogen activator inhibitor may be useful when the desired treatment involves areas or organs accessible by topical application, such as the epidermis, surface wounds, or areas made accessible during surgery. Carriers for topical administration of a Y. pestis plasminogen activator inhibitor described herein include, but are not limited to, mineral oil, liquid petroleum, white petroleum, propylene glycol, polyoxyethylene polyoxypropylene compounds, emulsifying wax, and water. Alternatively, a topical composition comprising a Y. pestis plasminogen activator inhibitor as described herein may be formulated with a suitable lotion or cream that contains the inhibitor suspended or dissolved in a suitable carrier to promote absorption of the inhibitor by the upper dermal layers without significant penetration to the lower dermal layers and underlying vasculature. Carriers that are particularly suited for topical administration include, but are not limited to, mineral oil, sorbitan monostearate, polysorbate 60, cetyl esters wax, cetearyl alcohol, 2-octyldodecanol, benzyl alcohol, and water. A Y. pestis plasminogen activator inhibitor may also be formulated for topical application as a jelly, gel, or emollient. Topical administration may also be accomplished via a dermal patch.

Persons skilled in the field of topical and transdermal formulations are aware that selection and formulation of various ingredients, such as absorption enhancers, emollients, and other agents, can provide a composition that is particularly suited for topical administration (i.e., staying predominantly on the surface or upper dermal layers with minimal or no absorption by lower dermal layers and underlying vasculature) or transdermal administration (absorption across the upper dermal layers and penetrating to the lower dermal layers and underlying vasculature).

Pharmaceutical compositions comprising a Y. pestis plasminogen activator inhibitor as described herein may be formulated for nasal administrations, in which case absorption may occur via the mucous membranes of the nasal passages or the lungs. Such modes of administration typically require that the composition be provided in the form of a powder, solution, or liquid suspension, which is then mixed with a gas (e.g., air, oxygen, nitrogen, or a combination thereof) so as to generate an aerosol or suspension of droplets or particles. Inhalable powder compositions preferably employ a low or non-irritating powder carrier, such as melezitose (melicitose). Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Pharmaceutical compositions described herein may be packaged in a variety of ways appropriate to the dosage form and mode of administration. These include but are not limited to vials, bottles, cans, packets, ampoules, cartons, flexible containers, inhalers, and nebulizers. Such compositions may be packaged for single or multiple administrations from the same container. Kits may be provided comprising a composition, preferably as a dry powder or lyophilized form, comprising a Y. pestis plasminogen activator inhibitor and preferably an appropriate diluent, which is combined with the dry or lyophilized composition shortly before administration as explained in the accompanying instructions of use. Pharmaceutical composition may also be packaged in single use pre-filled syringes or in cartridges for auto-injectors and needleless jet injectors. Multi-use packaging may require the addition of antimicrobial agents such as phenol, benzyl alcohol, meta-cresol, methyl paraben, propyl paraben, benzalconium chloride, and benzethonium chloride, at concentrations that will prevent the growth of bacteria, fungi, and the like, but that are non-toxic when administered to a patient.

Consistent with good manufacturing practices, which are in current use in the pharmaceutical industry and which are well known to the skilled practitioner, all components contacting or comprising a pharmaceutical composition must be sterile and periodically tested for sterility in accordance with industry norms. Methods for sterilization include ultrafiltration, autoclaving, dry and wet heating, exposure to gases such as ethylene oxide, exposure to liquids, such as oxidizing agents, including sodium hypochlorite (bleach), exposure to high energy electromagnetic radiation (e.g., ultraviolet light, x-rays, gamma rays, ionizing radiation). Choice of method of sterilization will be made by the skilled practitioner with the goal of effecting the most efficient sterilization that does not significantly alter a desired biological function of the Y. pestis plasminogen activator inhibitor or other component of the composition.

Additional embodiments and features of the invention will be apparent from the following non-limiting examples.

Y. pestis Cell Surface Plasminogen Activator and Related Virulence Factors

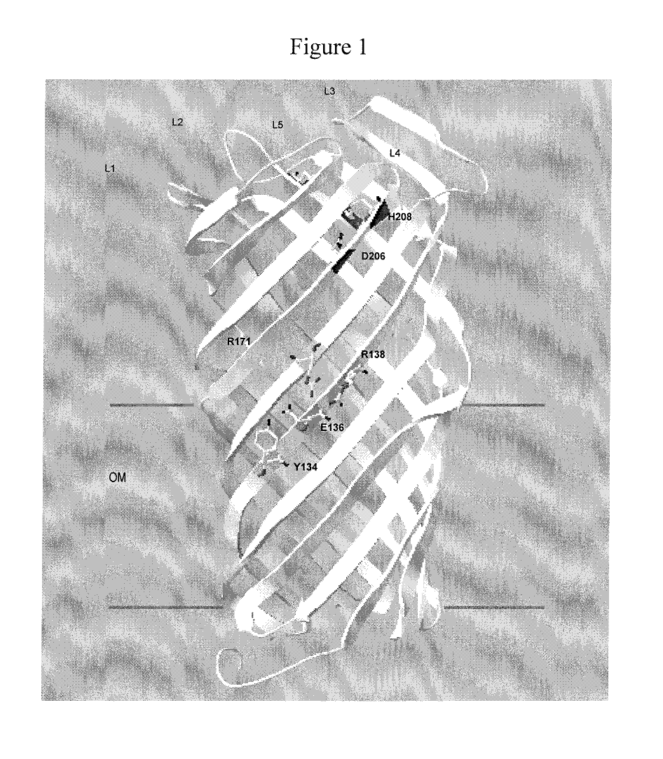

Y. pestis cell surface plasminogen activator (Pla) is a 292 amino acid (mature form) protein of the six-membered “omptin” family of β-barrel outer membrane proteases. Initially, the omptins were considered serine proteases that lacked the classical serine protease consensus sequence. However, the crystal structure of E. coli OmpT, the prototype member of the family revealed that the proposed catalytic residues Ser-99 and His-212 are too far apart to function together (McCarter, J. D., et al., J. Bacteriol., 186: 5919-25 (2004); Vandeputte-Rutten, L., et al., Embo. J., 20: 5033-9 (2001)). In addition, classical serine protease inhibitors such as DFP do not inhibit or inhibit omptins very poorly. A water molecule activated by a His-Asp and an Asp-Asp pair appears to act as a nucleophile in place of the γ-oxygen of serine observed in serine proteases (Kramer, R. A., et al., FEBS Lett., 505: 426-30 (2001); Vandeputte-Rutten, L., et al., Curr. Opin. Struct. Biol. 12: 704-8 (2002)). A model of Pla based on the OmpT structure reveals the basic arrangement of the protein in the membrane with the active site residing in exposed loops outside the cell (see, FIG. 1) (Kukkonen, M., et al., Mol. Microbiol., 51: 215-25 (2004)). Pla has been demonstrated to activate plasminogen (Sodeinde, O. A., et al., (1992) op. cit.) and degrade α2AP (Kukkonen, M., et al., Mol. Microbiol., 40: 1097-111 (2001)), T7 RNA Polymerase, and cationic antimicrobial peptides (Galvan, E. M., et al., Infect. Immun., 76: 1456-64 (2008)), as well as facilitate (adhesion to and) invasion of cells (Lahteenmaki, K., et al., FEBS Lett. 504: 69-72 (2001)).

Most omptin family members found in recognized pathogens and specifically tested in infection models to date are virulence factors (Hritonenko, V., et al., Mol. Membr. Biol. 24: 395-406 (2007)). Those most closely related to Pla include Salmonella enterica PgtE (72% aa identity; 83% similarity), E. coli OmpT (47% aa identity; 63% similarity), and Shigella SopA (also called IcsP) (41% aa identity; 58% similarity). Y. pestis employs Pla as a non-redundant part of its arsenal of virulence factors to avoid and/or eliminate the innate immune response to infection. Related omptins, E. coli OmpT and Salmonella PgtE, appear to accomplish a similar goal by proteolysis of antibacterial peptides of the innate immune system (Bader, M. W., et al., Mol. Microbiol. 50: 219-30 (2003); Guina, T., et al., J. Bacteria, 182: 4077-86 (2000); Stumpe, S., J. Bacteria 180: 4002-6 (1998)). The Shigella omptin, SopA, forms of which are present in three oral-fecal diarrhea and dysentery pathogens, enteroinvasive E. coli, Shigella flexneri and Shigella dysenteriae, plays a role in intra- and intercellular movement of the pathogen in human host cells (Monack, D. M., et al., Cell Microbiol. 3: 633-47 (2001); Wing, H. J., et al., J. Bacteria 186: 699-705 (2004)).

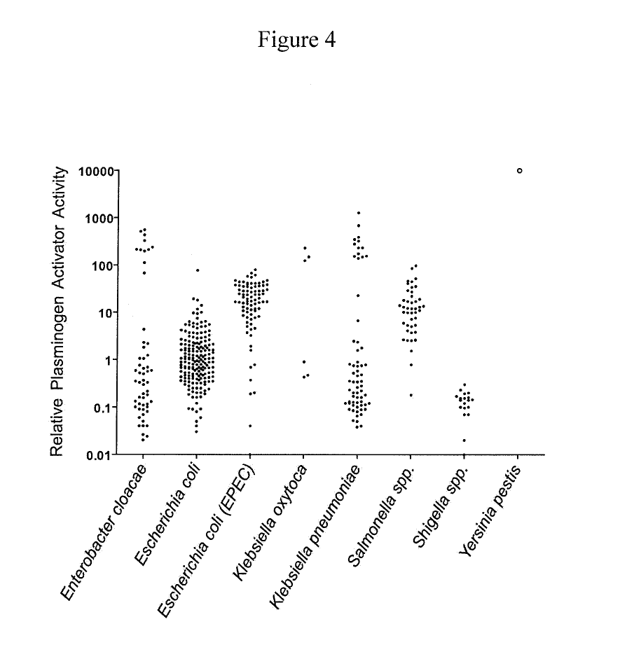

Drugs with the ability to inhibit more than one of these related virulence proteases may have broad utility in the clinic as well as in biodefense. We have surveyed more than 700 clinical isolates of 41 species of Enterbacteriacea for Pla-like activity using plasminogen as substrate (see, FIG. 4). Results show that this activity is widespread, and present in species not previously known to have omptin members, including Enterobacter cloacae and Klebsiella pneumoniae. We sequenced the omptin member from a high-activity E. cloacae isolate, and found it to be more closely related to Pla (79% identity) than any other family member.

In summary, studies indicate that omptin members (1) are very often virulence factors and are probably important in species for which their contribution is not yet recognized; (2) have plasminogen activator activity which, while readily detectable, declines as they diverge in similarity from Pla; and (3) they serve different functions in different pathogens and may attack host proteins, or as in the case of Shigella, process bacterial membrane proteins.

The importance of Pla to the virulence of Y. pestis is indisputable, but the precise mechanisms by which it performs its role are complex. Comparisons of the pathology of infections of animal models with wild-type Y. pestis and with Pla-deficient Y. pestis strains have established the following points (see, Table 1).

TABLE 1
Consequences of Pla-deficiency on Y. pestis Infections of Mice

| | Route of Administration of Y. pestis | |
|---|---|---|
| Bacterial | Subcutaneous | Intranasal |
| Genotype | [see, Sodeinde, O. A., et al., (1992) op. cit.] | [see, Lathem, W. W., et al., (2007) op. cit.]. |
| Y. pestis Pla+ | Local mass of bacterial growth | Bacterial growth by 6 logs in 2 days |
| (wild-type) | Few inflammatory cells | Rapid progressive disease with |
| | Disseminated infection (liver & | edema in the lungs, tissue |
| | spleen) | destruction, hemorrhage |
| | Death within 3-4 days | Dissemination to lung & spleen by |
| | | 2-3 days |
| | | Death of all mice by 4 days by |
| | | pneumonia |
| Y. pestis Pla− | Local mass of bacterial growth, | Very little bacterial growth or |
| | but few free bacteria | edema in the lungs; restricted foci |
| | Many inflammatory cells | of inflammation |
| | (neutrophils), especially at | Non-progressive lung infection |
| | margin of edematous tissue | Slower dissemination to lung & |
| | Poor dissemination | spleen |
| | No deaths observed at end of | Death of ½ of mice by 7 days, by |
| | expt. (21 days) | septicemia rather than pneumonia |

First, it is important to note that the consequences of the loss of Pla on pathology depend on the route of infection and the type of ensuing disease. The route of administration of Y. pestis may be subcutaneous (including flea bite) producing bubonic plague, intranasal resulting in pneumonic plague, or intravenous, causing septicemia directly. Loss of Pla results in a million-fold increase in LD₅₀ for subcutaneously infected mice (10⁷ vs. 50 bacterial cells) (Sodeinde, O. A., et al., (1992) op. cit.). In contrast, death is not prevented, but is significantly delayed for the pneumonic and septicemic cases in Pla-deficient infections (Lathem, W. W., et al., (2007) op. cit.; Sebbane, F., et al., (2006) op. cit.). One clearly established activity of Pla is activation of plasminogen. In solid tissue, where the effect has been most thoroughly studied, this results in lack of local neutrophil accumulation, preventing microabscess formation and allowing unfettered dissemination of the bacteria (see, Example 1; Degen, J. L., et al., J. Thromb. Haemost., 5(Supp1)1: 24-31 (2007); Sodeinde, O. A., et al., (1992) op. cit.)). In the lung, plasminogen activation is apparently also important (Lathem, W. W., et al., (2007) op. cit.), but other mechanisms including degradation of antibacterial peptides may also come into play (Galvan, E. M., et al., (2008) op. cit.). In all cases, loss of Pla provides a significant benefit to the host.

The discovery of small molecule Pla inhibitors provides a therapeutic approach to mimic the genetic deletion of pla and slow down the progression of disease, permitting appropriate antibacterials to be selected.

The basic experiments comparing Pla-proficient and Pla-deficient Y. pestis strains in bubonic and pneumonic plague models have been elaborated further in two ways. First, mouse infection experiments have been done with intranasal administration of a Y. pestis strain engineered to place pla gene expression under regulated control of anhydro-tetracycline (ATC) (Lathem, W. W., et al., (2007) op. cit.). In these experiments, removal of the inducer of pla expression one day after infection still provided a significant delay in death, suggesting that administration of Pla inhibitors as late as one day after infection would expand the window during which antibiotics could be administered as therapy.

As encouraging as these results are, it is worth noting that turning off gene expression is a “slow” way to reduce Pla amounts because existing protein has to be degraded or diluted out; a drug would work faster by immediately inhibiting the activity of Pla. Consequently, discovery of small molecule inhibitors enables a true proof of principle for the efficacy of this therapeutic approach. Second, Degen et al. (Degen, J. L., et al., (2007) op. cit.) have described the effects of wild-type Y. pestis infection of mice lacking plasminogen or fibrinogen. Subcutaneous infection of wild-type mice with Y. pestis resulted in widespread foci containing massive numbers of free bacteria with little inflammatory infiltrate. However, loss of Pla or loss of host plasminogen resulted in the accumulation of robust inflammatory cell infiltrates at sites of infection and greatly improved survival. Fibrin(ogen) deficiency of the host effectively eliminates the survival benefits of deletion of either Pla or host plasminogen. Plasminogen and fibrinogen are extremely effective modulators of the inflammatory response in vivo and critical determinants of bacterial virulence and host defense.

In summary, Pla was selected as a therapeutic target in view of following considerations:
Novel mode of action. Loss of Pla accelerates the proinflammatory response of the innate immune system in infection and significantly delays the rate of death. Consequently, therapeutics that inhibit Pla are expected to prolong the time for therapeutic intervention ensuring adequate time for determination of drug susceptibility of the infectious strain and for antibiotic therapy. Pla-inhibitory drugs will be administered prior to and in combination with appropriate antibacterials and will facilitate therapy.
Feasibility
   Accessibility to small molecules—location of the target on the cell surface is particularly important for Gram(−) species such as Y. pestis because the outer membrane presents an extra barrier to drugs which must act cytoplasmically.
   Knowledge base of protease inhibitor discovery and design. The successful discovery and therapeutic use of drugs which inhibit proteases argues that this class of targets is particularly “druggable”.
Selectivity. No members of the omptin family are mammalian proteases, and a BLASTP search with Pla reveals no significant hits in the human refseq database. Consequently, inhibitors of Pla and other omptins are unlikely to exhibit target-based toxicity.
Target Structure. Successful crystallization and structure determination of OmpT led us to pursue structural studies of Pla. Diffraction quality crystals of Pla have now been obtained (JG, unpublished collaboration at U. Mass. Medical School), and x-ray diffraction will be done soon, but no detailed Pla structure is available yet.
Spectrum. Salmonella PgtE, E. coli OmpT, and Shigella SopA/IcsP possess amino acid sequence similarities to Pla well above 50%; so, Pla inhibitors may also be effective inhibitors of one or more of these virulence factors in other Gram-negative pathogens.

### Example 1

### Confirmation of Link between Pla and Virulence

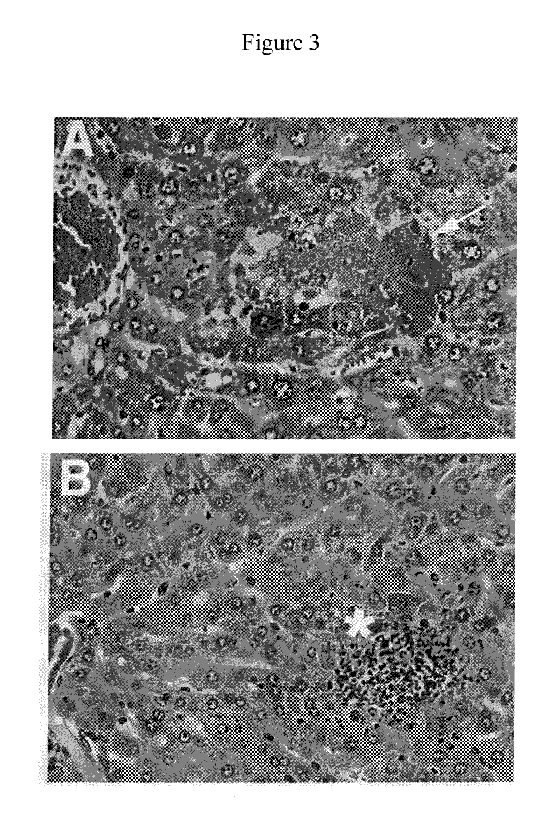

In subcutaneous infection of mice, Pla plays a key role in virulence (Sodeinde, O. A., et al., (1992) op. cit.): Pla-deficient mutants are reduced in virulence by a factor of a million. This loss of virulence is accompanied by inability to prevent the rapid accumulation of inflammatory cells, particularly neutrophils, at foci of infection. For preliminary efficacy testing of Pla inhibitors in vivo, mice are infected intravenously with 1,000 Y. pestis, and at two days post-infection are sacrificed and their livers examined histologically. At the time of infection, some bacteria are deposited in the liver and the extent of inflammatory cell infiltration at the sites of colonization is readily seen in hematoxylin-eosin (H+E) stained sections (see, FIGS. 3A & 3B). When wild-type bacteria are used, masses of bacteria are seen packed in liver sinusoids (see, FIG. 3A, arrow) but virtually no inflammatory cells are present, a remarkable demonstration of the ability of Y. pestis to suppress and evade innate immune defenses. In contrast, when Pla-deficient bacteria are used, few free bacteria are visible in the liver. Instead, microabscesses consisting of masses of inflammatory cells surrounding the bacteria are observed (see, FIG. 3B, asterisk). The identical phenotype is displayed when some other functions required to control local inflammation are compromised. For example, we recently showed that forcing Y. pestis to produce TLR4-stimulating LPS, in contrast to the inactive form usually made, has an identical effect (Montminy, S. W., et al., (2006) op. cit.). We have also shown that the ability of Pla-producing Y. pestis to suppress local inflammation is dependent on host plasminogen (Degen, J. L., et al., (2007) op. cit.): in plasminogen-deficient mice, wildtype Y. pestis is unable to prevent microabscess formation, and this is correlated with enhanced resistance of the mice to infection. We also found that fibrinogen-deficient mice were unable to form microabscesses in response to Y. pestis regardless of their plasminogen status or the Pla status of the bacteria, and that these mice were exquisitely sensitive to infection. Taken as a whole, these data suggest a model in which fibrin is crucial to microabscess formation during Y. pestis infection, and that Pla destroys locally deposited fibrin via activation of plasminogen. The hypothesis that we currently favor is that incoming inflammatory cells avoid intoxication via T3SS by binding to the fibrin close to—but not in contact with—the bacteria. This allows them to produce pro-inflammatory cytokines, and in particular neutrophil chemokines, that attract increasing numbers of cells to the lesion.

### Example 2

### Broad Distribution of Pla Activity Among Enterobacteriaceae

We have examined octylglucoside extracts of 735 bacterial clinical isolates representing 41 species for plasminogen activating activity. The results (see, FIG. 4) reveal that while none produces as much activity as Y. pestis on a per cell basis, many strains and species do produce significant Pla-like activity. Most are known to carry omptin family members, but our findings suggest that the family is larger than previously known and includes Enterobacter and Klebsiella. In fact, we cloned and sequenced the omptin produced by the E. cloacae strain with the highest level of activity, and found it more closely related to Y. pestis Pla than any other omptin described to date. Of course, the majority of the 735 strains did not produce significant Pla-like activity, indicating that this activity is not due to non-specific proteases.

### Example 3

### Preparation of Recombinant Pla Produced in E. coli

Although it is possible to purify the enzyme safely from attenuated Y. pestis strains, we have done the bulk of our work with Pla isolated from an E. coli strain engineered to express the enzyme at very high levels. In this strain, BL21(pBSpla), the pla gene (Genbank Accession number AAA27667) is driven by its native promoter but contained in a high copy number plasmid (pBlueScript, Agilent Technologies, Wilmington, Del.). Pla comprises about 40% of total outer membrane protein in this strain. Remarkably, this high level of expression has minimal effects on growth, and the plasmid is easily maintained via ampicillin selection. This level of expression is about 10-fold higher than that observed in Y. pestis. BL21(pBSp/a) also lacks OmpT, the E. coli Pla homolog.

### Example 4

### Activity and Kinetic Studies

We have conducted extensive kinetic studies of plasminogen activation by Pla contained in intact bacteria, in purified membranes, and in soluble form. While there is no discernable difference between behavior of the enzyme in whole cells and in purified outer membranes, the purified soluble enzyme behaves very differently in four respects: (a) the membrane-bound form is more stable; (b) the soluble form has a pH optimum at 5.7 and is inactive at physiologic pH; (c) the soluble form has optimum activity at non-physiologic ionic strength (600 mM); and (d) kinetic behavior is grossly altered in the soluble form (Kₘ of 1.2 mM vs. 95 nM for the membrane-bound form). Taken together, these observations argue strongly for use of membrane-bound Pla—either in isolated membranes or in intact bacteria—during screening for Pla inhibitors. The stability of the membrane-bound enzyme, the ability to conduct the assays at physiological pH and ionic strength, and retention of the physiologic interaction of plasminogen and plasmin with Pla during screening are major advantages that enhance the likelihood of identifying useful inhibitors.

### Example 5

### Pla Specificity

The relatively high specificity of Pla for protein substrates suggests that the Pla-plasminogen interaction involves an extended active site. One approach we have taken to determining Pla specificity with respect to P1 and P1′ residues (the residues immediately flanking the active site), is to compare the relative effectiveness of a variety of peptides as competitive inhibitors of plasminogen activation. The most effective of these, along with their Kᵢ values (mM) are Arg-Lys (0.9), Lys-Lys (4.5), Arg-Val (4.8), Arg-Gly (5.2), Lys-Gly (5.6), and Lys-Val (8.4). All other dipeptides tested had Kᵢ above 15 mM. The sequences of these dipeptides with low Kᵢ correspond well with the few cleavage sites identified in protein substrates of Pla. For example, Pla activates plasminogen by cleaving the Arg-Val pair attacked by other plasminogen activators. Thus, Pla strongly prefers a basic residue at the P1 position, and either a basic residue or Val or Gly at P1′. However, the specificity of membrane-bound Pla for proteins is highly restricted. For example, the failure of Pla to attack the complement components C3, C4 and C5, all of which are readily cleaved by trypsin, indicates that substrate recognition is likely to involve an extended recognition site and be dependent on tertiary structure.

### Example 6

### Protease Inhibitors and Pla

A variety of commercial protease inhibitors were tested against Pla with entirely negative results except in the case of reactive species like DFP, which cause inhibition only at high concentrations at which many residues, rather than primarily active site serines, are affected. Metal chelators including EDTA and EGTA actually enhance activity, suggesting that divalent cations may be inhibitory. This effect might also be mediated by the destabilization of Pla containing membranes via extraction of divalent cations. Importantly, the aspartyl protease inhibitor pepstatin also has no inhibitory effect, consistent with the hypothesis based on crystal structure of OmpT, that Pla and other omptins are not “traditional” aspartyl proteases, but combine aspects of both serine and aspartate proteases, likely utilizing as a nucelophile a bound water molecule activated by asp-asp and asp-his pairs (Kramer, R. A., et al., (2001) op. cit.; Vandeputte-Rutten, L., et al., (2002) op. cit.; Vandeputte-Rutten, L., et al., (2001) op. cit.).

### Example 7

### Development of a High Throughput Screen for Inhibitors of Pla

Human Glu-plasminogen (Glu-Plg), the major form in blood, was used as the substrate for inhibitor screening because it is the most physiologically relevant and can be readily adapted to high-throughput techniques. D-Val-Leu-Lys-p-nitroanalide (trade name S2251) is a chromogenic plasmin substrate that has been used successfully by many researchers to measure rates of plaminogen activation (Koh, S. C., et al., Immunol. Cell Biol., 67 (Pt 3): 197-203 (1989), Latallo, Z. S., et al., Haemostasis, 7: 150-4 (1978)). We have found it to be immune to hydrolysis by Y. pestis Pla, and E. coli BL21(pBSpla) cells expressing membrane-bound Pla. It may be used to measure Pla plasminogen activator activity. Briefly, Pla (as membranes or intact bacteria) is mixed with S2251, and the reaction is started by addition of plasminogen. In this coupled assay, color development due to the substrate cleavage by plasmin created in the reaction follows a parabolic trajectory because the amount of plasmin is continuously increasing.

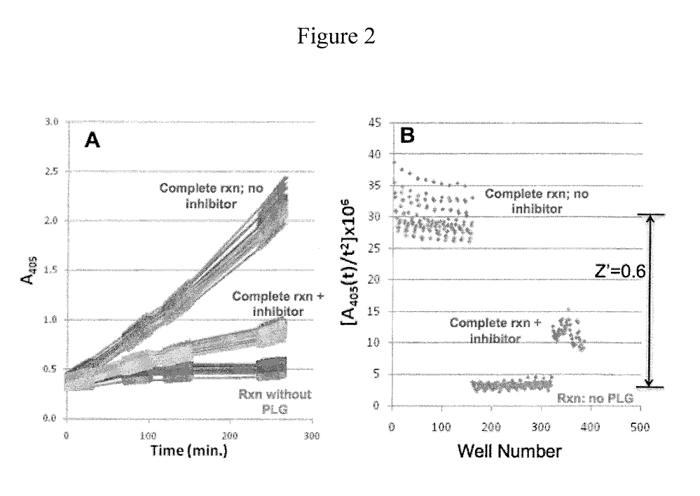

Assays were performed in a 100 μL volume containing 3 nM Pla, 5 mM S2251, 160 nM human Glu-Plg, 50 mM Tris, and 0.01% Tween 80 at pH 7.4. We grew E. coli BL21(pBSp/a) cells in LB+1 mg/ml ampicillin overnight, adjusted the OD₆₀₀ to 1 in the morning, centrifuged and resuspended the cells in the same volume of buffer. When 4 μL of this cell suspension was used in a 100 μL reaction, there were sufficient cells (˜4×10⁶) and associated Pla to cleave S2551 and generate a continuously increasing A₄₀₅ signal for over 260 min at room temperature in a kinetic study of 16 wells each of positive and negative controls and inhibitor (see, FIG. 2, panel A). The number of cells was low enough to eliminate any contribution to the A₄₀₅ reading due to light scattering. Note that the stability of the signal gain and the dependency on added Glu-Plg throughout 260 min under these conditions indicates that the few E. coli cells present are not significantly degrading the chromogenic substrate (S2251). We used 16 mM NH₂-Lys-Val dipeptide as a non-specific inhibitor to demonstrate detectable inhibition. All wells contained 2% DMSO, which had no detectable effect on the assay, but allowed us to test concentrations of compounds up to 50 μM in the screen (i.e., 50-fold dilution from the 2.5 mM master storage plates). Some library compounds were colored, but at 50 μM, we confirmed that the color intensity was too low to interfere with the A₄₀₅ readings.

Under these conditions, the positive and negative control A₄₀₅ signals exhibited a signal-to-background of ˜8 at 120 min (see, FIG. 2, panel A). In addition, they reached a Z′-factor (Zhang, J. H., et al., (1999) op. cit.) value of 0.6 by 120 min and remained between 0.62 and 0.65 for another 2 hr. A screen with a Z′-factor>0.5 is considered excellent (Zhang, J. H., et al., (1999) op. cit.). The Z′-factor is a screening window coefficient that is defined as the ratio of the positive and negative control separation band to the signal dynamic range of the assay. The stability of the Z′-factor means that readings can be taken any time between 2 hr and 4 hr as long as they are all taken consistently at the same time.

Next, a complete 384-well microplate assay was prepared and an endpoint reading at 120 min (see, FIG. 2, panel B) was taken. The NH₂-Lys-Val inhibitor was added first to the appropriate wells in the plate. Then, a mixture of buffer, S2251, and BL21(pBSpla) cells was added to each well with the Wellmate reagent dispenser. Finally, the screen was initiated by addition of a mixture of buffer and Glu-Plg, also with the Wellmate. Readings were taken in an Envision microplate reader. The Z′-factor was 0.65 and the S/B was 9.2 (see, FIG. 2 panel B). Average inhibition from the 16 mM NH₂-Lys-Val was 60% and was highly statistically significant (>6 standard deviations below the negative control complete reaction).

About 1 min is required to add reagents to a single 384-well plate with the Wellmate and a similar time is required to read it in the Envision. For the high throughput screen, compound addition was done robotically with the Sciclone liquid handling robot and Twister plate handler. Then, wells were loaded with buffer, S2251, and cells. Plates were handled at a comfortable pace at this stage because this mixture is quite stable for hours. The HTS is initiated by adding the Glu-Plg and buffer mixture to 120 plates (38,400 compounds) and moving the stacks to the Envision for reading as soon as reagent addition is complete, resulting in a 120 min incubation at room temperature.

### Example 8

### Optimization and Pilot Screen for Inhibitors of Membrane-Associated Y. pestis Pla

The HTS (Example 7) was applied to 2,000 compounds in a pilot screen to ensure that it was suitable for high throughput applications to large chemical libraries. Recombinant E. coli cells overexpressing Y. pestis Pla (see, Example 3) were used. Washed cells were used as the source of enzyme because the properties of purified membrane-free preparations of Pla are altered and because recombinant protein is produced so abundantly that very few cells were needed per assay. This eliminated any light scattering artifacts in the assay. Overnight saturated cultures of the recombinant cells grown with ampicillin selection for the plasmid provided ample Pla. The concentration of each component in the assay was optimized to maximize the separation band between positive and negative controls as evaluated by the Z′ factor (Zhang, J. H., et al., (1999) op. cit.).

### Optimization of the Chromogenic Plasminogen Activation Assay

Parameters of the HTS assay were set as follows: 10 nM Glu-plasminogen (Glu-Plg; American Diagnostica, Inc.), which is equivalent to 0.8 μg/ml or 0.08 μg in a 100 μl assay; 50 mM Tris-HCl pH 7.4, 0.01% Tween 80; washed cells of E. coli strain BL21(pBSpla) to give an effective concentration of 5 nM Pla in the assay; and 200 μM S2251 (H-D-Val-Leu-Lys-pNA 2HCl from DiaPharma Group, Inc.). The assay was performed at room temperature, and hydrolysis of S2251 was monitored by A₄₀₅. For this coupled assay, the activity of Pla is proportional to the derivative of the ΔA₄₀₅ curve vs. time, or more simply to the A₄₀₅ at time t divided by t². Because it requires the least manipulation, this single-stage endpoint assay is best suited for a high throughput screen (HTS). For HTS, the Pla-containing cells were mixed with S2251 and this cocktail was added to the assay plates containing chemical compounds by using the Wellmate dispenser. The reaction was started by adding Glu-Plg and read A₄₀₅ after a fixed time incubation of about 75 minutes. It is important to add Glu-Plg last because even trace amounts of contaminating plasmin will cleave S52251 and generate high backgrounds if left for long time periods. By contrast, Pla-containing E. coli cells have no effect on S2251 over long incubation periods. The screen tolerated DMSO at concentrations up to at least 2%, which will permit addition of screening compounds at concentrations up to 50 μM (50-fold dilution of master plate concentration of 2.5 mM). Here, the signal:background and the Z′ value were maximized and optimized the concentrations of reagents were optimized. Reconfiguration of the chemical libraries to 384-well format was done by the Sciclone liquid handling robot as compounds were added to the assay plates. Several plates filled with ½ positive and ½ negative controls were run, and the Z′ value under each condition examined was determined (Zhang, J. H., et al., (1999) op. cit.). Conditions which provided a Z′ of >0.6 were considered acceptable.

### Pilot Screen to Assess Screening Conditions.

The optimized assay configuration was tested in a pilot screen of ˜2,000 compounds at 2-3 different concentrations. Controls were included in each plate—the first two columns of the 384-well microplate for 0% inhibition (DMSO only, maximal signal=negative control) and the last column for zero Pla activity (no Glu-Plg) (maximal inhibition=positive control). Assay plates received recombinant Pla-containing cells and compounds to be tested according to the protocol determined in the assay development phase above. The data obtained from this screen were used to determine variation (% CV), the Z′ value, and to identify any problems with the assay which require resolution before HTS began. The data from the pilot screen was used to determine the compound concentration for the screen (probably in the range of 25-50 μM) in order to establish a hit rate between 0.1% and 1%. The criteria for designating a compound as a hit was determined following the pilot screen; however, an inhibition of ≧50% and a Z-score>3 will likely be suitable. The Z-score for inhibition by each sample represents the number of standard deviations below the negative control A₄₀₅ value (i.e., maximal signal) that is observed for the sample. The Z-score for each sample will be derived by subtracting the sample A₄₀₅ value from the mean negative control A₄₀₅ value and dividing the difference by the negative control standard deviation.

### Example 9

### Screen the Library to Identify, Deconvolute, and Confirm Pla Inhibitors

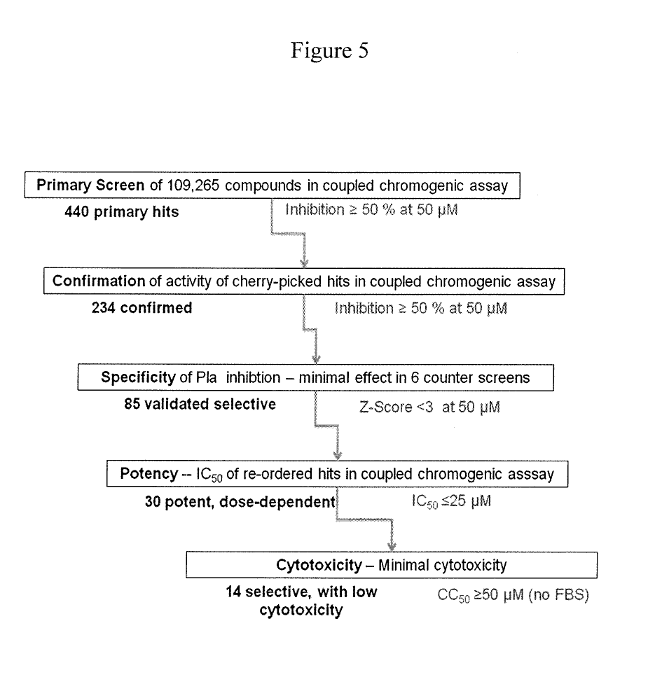

The high throughput Pla screen developed in the preceding examples was applied to a library of discrete small molecules and natural products in order to identify compounds having potent inhibitory activity against membrane-associated Y. pestis Pla. A flow chart setting forth the screening and hit analysis is set forth in FIG. 5. Hits from the screen were confirmed by re-assay, by establishing that they inhibit Pla and not the assay coupling enzyme plasmin, by demonstrating their concentration-dependent inhibition (IC₅₀), by eliminating non-specific inhibitors that inhibit other proteases (e.g., human tPA, uPA, cathepsin D, cathepsin E, and HIV-1 protease), and by eliminating inhibitors that are cytotoxic to HeLa cells in culture (CC50>50 μM).

### Compound Libraries and Sample Handling

A compound repository of −430,000 discrete chemical samples, which encompasses approximately 300 chemotypes was built. Compounds were obtained, as follows: 20,000 from Maybridge (Cornwall, UK), 2,000 from Microsource Discovery Systems, Inc. (Gaylordsville, Conn.), 20,000 from Chemical Diversity Labs (San Diego, Calif.), 70,000 from ChemBridge Corp. (DIVERSet™; San Diego, Calif.), and 12,000 from TimTec, Inc. (Newark, Del.), including about 1,400 discrete purified natural products. Compounds were selected in the molecular size range of 200 to about 500 Da. Compounds were evaluated using numerous chemical filters, including Lipinski's ‘Rule of 5’ (Lipinski, C. A., J. Pharmacol. Toxicol. Methods, 44: 235-49 (2000)), and filters designed to remove unwanted and known cytotoxic fragments. The library was screened using the primary HTS assay.

### Application of the Primary Pla HTS Screen

Compounds in the library were examined in 384-well format against the cell-associated Pla HTS using the conditions and the hit definition (inhibition of ≧50% and a Z-score>3) established in Example 8. Screening library compounds are stored in 96-well master plates at 2.5 mM in 100% DMSO at −20° C. Master plates were thawed, and 50 μM of compound were added to the assay plates by means of a Sciclone ALH 3000 liquid handling robot (Caliper, Inc.) and a Twister II Microplate Handler (Caliper, Inc.), at the same time, combining 4×96-well source plates into one 384-well assay plate. The screening plates contained positive and negative controls in the first and last columns as described Example 8. Using the single-stage HTS of Example 8, a volume of assay mix (cell-associated Pla+S2251) was added to each well of the screening plates by means of a Wellmate Microplate reagent dispenser (ThermoFisher/Matrix). The reaction was initiated by the addition of Glu-Plg (also by Wellmate), and plates were incubated at room temperature for 75 min. The A₄₅₀ was read by an Envision Multilabel Reader (PerkinElmer, Inc.). The speed of the Wellmate and Envision are similar; so, the time between Glu-Pig addition and A₄₅₀ reading was sufficiently constant for each well.

Raw data generated by the plate reader were processed as follows: A₄₀₅ data were captured and analyzed in a semi-automated procedure by relating the plate serial number to the database entry, associating the numerical readout to each compound entry, and calculating the % inhibition and Z-score. In addition, a Z′-factor calculation (Zhang, J. H., et al., (1999) op. cit.) was performed on each plate based on the positive and negative controls; Z′ factor values of ≧0.6 were considered adequate, and data from compounds in that plate were accepted into the database. All screening data, including the % inhibition, Z-score, and confirmation/validation data such as the 50% inhibitory concentration (IC₅₀) and the counter-screen results were stored in one central database (ChemBioOffice, CambridgeSoft, Inc., MA).

### Example 10

### Hit Confirmation, Verification and Deconvolution

Compounds that satisfied the criteria for designation as primary hits were subjected to a 3-step confirmation process (see, FIG. 5). First, primary hits were cherry-picked from stock plates into a confirmation stock plate and replicated to produce a set of 4 confirmation assay plates. The 4 confirmation assay plates were used in the primary screening assay to generate 4 new data points for each compound. A confirmed hit was required to display inhibition>50% and a z-score>3 in at least 3 of the 4 replicated assays. Second, confirmed hits were assayed for plasmin inhibitory activity to ensure that hits do not inhibit the coupling enzyme rather than the plasminogen activator, Pla. Third, confirmed hits were examined for concentration-dependent activity in the Pla assay, and an IC₅₀ was determined to rank the potency of each.

Confirmed hits with favorable IC₅₀ values (i.e., ≦10 μM) were re-synthesized and/or re-ordered from a different batch. Their purity and mass were verified, and they were tested as follows.

### (A) QC and Medicinal Chemistry Evaluation

Compounds were subjected to analysis by LC-MS in order to establish that they are ≧95% pure and of the correct molecular weight. Compounds that failed this analysis were abandoned. In addition, compounds that are promiscuous (active in many screens with little selectivity) or compounds that are too reactive chemically, such as alkylating agents or acylating agents, were excluded from further analyses due to potential toxicity.

### (B) Primary and Secondary Assays and Counter-Screens

Re-ordered and/or re-synthesized confirmed hits with verified purity and mass and IC₅₀ values≦10 μM were retested in the primary assay to confirm activity, then tested in the secondary assays (discussed below) to establish that they inhibit the range of physiological Pla activities and to determine their species spectrum of activity. As an initial test of spectrum, we examined compounds for inhibition of the Pla-like activity detected in 7 Enterobacteriaceae species using the whole cell assay described in Example 2 (see, FIG. 4). O-antigen polysaccharides can interfere with plasminogen-based whole cell assays because they hinder access of this substrate to the omptins. To avoid this complication and ensure a valid comparison, these omptins will be expressed in E. coli strain BL21, the same strain used to express Pla in the HTS assay. Clones expressing omptins from E. coli, Enterobacteria cloaceae, and Salmonella typhimurium are already on hand in the Goguen lab. We will also investigate whether published inhibitors of the HIV aspartyl protease, such as amprenavir, indinavir, ritonavir, nelfinavir, or lopinavir, exhibit potency vs. Pla.

Finally, potent hits were tested for inhibition of a panel of human proteases (see, Example 11) to demonstrate selectivity for Pla inhibition. Primary hits that pass the secondary assay and concentration dependence tests were considered validated hits. It is recognized that a few Pla inhibitors may exhibit some inhibition toward human proteases, and therefore compounds are prioritized based on the maximal selectivity (at least 10-fold more potent vs. Pla than vs. other proteases) to ensure that the most selective inhibitors are studied.

### (C) Minimal Inhibition of Mammalian Cells

Cytotoxic concentration, CC₅₀, of the compound was determined against cultured mammalian cells in the absence of serum in order to ensure that binding to serum proteins did not mask cytotoxicity. This procedure involves incubation of HeLa cells in culture with serial dilutions of the Pla inhibitor compounds. The CC₅₀ is defined as the concentration of compound that inhibits 50% of the conversion of the tetrazolium salt MTS to formazan. While HeLa cells are easy to grow and maintain, they may not accurately represent normal human cells. Therefore, any compounds that show low or no toxicity against HeLa cell cultures will be tested further against MRC-5 and WI-38 cells (both human diploid fibroblast lines, ATCC# CLL-171 and CCL-75, respectively). Candidates for further development will be required to display an in vitro selectivity index (CC₅₀/IC₅₀) of 5 or greater.

### (D.i.) Acute Toxicity in Mice.

The goal of these studies is to determine the MTD and assess toxicity of test compounds after single doses in mice. Groups of 5 mice (female, Swiss-Webster, 20-25 g each) will be treated with increasing doses of test compounds in a suitable vehicle by i.p. administration. Five doses of test compound will be given, covering at least a 30-fold range of doses (e.g., 5 animals per dose plus 5 animals as vehicle controls). The doses are selected to include those expected to cause no adverse effect and those possibly causing major (life-threatening) toxicity. Observations of body weight, clinical signs, behavior and appearance changes, and survival will be made daily for 7 days. Doses that cause more than temporary discomfort will be noted and, as indicated by the severity of signs of toxicity, these animals will be humanely euthanized. This dose will be considered the minimal toxic dose, and the next lower dose will be designated the MTD. Once the MTD has been established, this dose will be administered to a second group of 5 mice to confirm that it is tolerated before use in efficacy experiments.

### (D.ii.) In Vivo Efficacy Studies in Mice.

Microabscess formation in the livers of i.v. infected mice will be used as a preliminary assay for evidence of in vivo efficacy because it is a sensitive indicator of the in vivo action of Pla and can be read within 48 hours of infection. As we will have little information regarding pharmacodynamics of the test compounds, the short duration of this assay is a distinct advantage. Groups of 5 mice will be given i.p. injections of compounds successful in the acute toxicity trials outlined above. These mice will then immediately receive an i.v. dose of 1,000 Y. pestis of virulent strain C092. At 48 hours post infection, the animals will be sacrificed, their livers harvested and fixed in 10% buffered formalin. The liver tissue will then be sectioned and stained with Hematoxylin-Eosin (H+E), and then scored by experienced examiners blind to the treatment. Controls groups will include animals receiving vehicle plus Y. pestis. To provide standards for comparison, additional control groups will be given i.p. saline injections, and then either mock-infected or infected with either CO92 or CO92(pla⁻). Scoring will be based on the size of lesions, and the extent of inflammatory cell infiltration. To provide an additional metric of efficacy, bacterial titers will also be obtained from the spleens of the mice at the time of sacrifice. Compounds yielding positive results (statistically significant increases in microabscess formation and/or decreased liver titers) will be retested to ensure validity of the results.

### Example 11

### Secondary Assays for Inhibitors of Y. Pestis Pla and Related Omptins

We validated assays for inhibition of degradation of α2AP and CAMPs and inhibition of cell invasion and counter-screen assays for inhibition of human tPA, uPA, and cathepsins D & E, all with S/B≧5, and a Z′≧0.5.

The purpose of this experiment was to provide assays to further verify the hits from the HTS by validating them as capable of inhibiting the full range of physiological functions attributed to Pla and to determine their spectrum of activity against related omptins, as well as to assess the selectivity of their anti-proteolytic activity. High throughput capability was not necessary because the assays were for validation of confirmed hits only (i.e., hits from the primary screen). These assays were used validate the re-ordered and/or synthesized compounds as inhibitors of the panoply of omptin functions.

### Degradation of Human α2-Anti-Plasmin (α2AP)

This assay is to confirm that inhibitors of Pla's plasminogen activator function also inhibit Pla's proteolysis of α2AP (Kukkonen, M., K. et al., Mol. Microbiol., 40: 1097-111 (2001); Suomalainen, M., J. et al., Adv. Exp. Med. Biol., 603: 268-78 (2007)). For α2AP cleavage assays, 2×10⁸ Y. pestis cells with Pla in the outer membrane are incubated with 5 μg of human α2AP (Calbiochem) at 37° C. in 100 μl of PBS with and without a range of inhibitor concentrations. Samples of 40 μl are taken for analysis at each to two time points, about 5 h and 20 h. The mixture is resolved on SDS-PAGE, transferred onto nitrocellulose membranes, and probed with rabbit anti-human α2AP IgG (diluted 1:750; Calbiochem) with detection by alkaline phosphatase-conjugated anti-rabbit IgG and the phosphatase substrate. Band intensities will be determined and used to calculate the degree of α2AP proteolysis and the IC₅₀ for each inhibitor tested. Alternatively, an α2AP inactivation assay to detect the loss of plasmin inhibiting activity of α2AP could be readily developed by titering the amount of α2AP remaining (50% inhibitory volume in an 52251 chromogenic plasmin assay) after treatment with Y. pestis cells with and without inhibitor present (Kukkonen, M., K. et al., (2001) op. cit.).

### Proteolysis of Cationic Antimicrobial Peptides (CAMPs)

The degradation of the human cathelicidin CAMP LL-37 by Pla likely contributes to the virulence of Y. pestis by aiding its escape from the innate immune response (Galvan, E. M., et al., (2008) op. cit.). Related omptins OmpT and PgtE have also been shown to cleave antimicrobial peptides (Hritonenko, V., et al., (2007) op. cit.). An assay may be performed to ensure that confirmed Pla inhibitors also inhibit the ability of Pla to degrade the CAMP LL-37 (Galvan, E. M., et al., (2008) op. cit.) and to examine the ability of the inhibitors to block proteoloysis by related omptins PgtE and OmpT. The C-terminal fragment of human cathelicidin, LL-37, (linear peptide of 37aa beginning with Leu-Leu; sequence: NH₂-LLGDFFRKSKEKIGKEFKRIVQRIKDFLRNLVPRTES-COOH (SEQ ID NO:1); purchased from Phoenix Pharmaceuticals, Inc., cat no 075-06) is subjected to incubation with Y. pestis cells carrying Pla in the same manner as described above for α2AP proteolysis assays. The Western blot is probed with a monoclonal antibody to LL-37 (Cell Sciences, Inc., Canton, Mass.; Clone 1-1C12; cat. No. HM2071). The IC₅₀ of each inhibitor will be calculated as described above from the Western blot band intensities.

A simpler assay with a surrogate CAMP substrate may be employed to determine whether the Pla inhibitors also inhibit OmpT and PgtE. Both omptin proteases have been shown to cleave T7 RNA polymerase, and activity can be measured conveniently in whole cell assays (Grodberg, J. et al., J. Bacteriol., 171: 2903-5 (1989); Grodberg, J., et al., J. Bacteriol., 170: 1245-53 (1988)). Intact bacterial cells expressing the proteases are incubated with T7 RNA polymerase (Epicentre Biotechnologies, Inc.)+/−Pla inhibitors, sedimented by centrifugation, and the supernatant analyzed directly by SDS-PAGE without Western blotting as described previously (Grodberg, J., et al., (1988), op. cit.). For E. coli OmpT, E. coli K12 laboratory strains are used, and for Salmonella PgtE, laboratory strains of S. typhimurium are used, or if more sensitivity is required, cloning and over-expression of the pgtE gene in E. coli B strain BL21, which does not carry the ompT gene, can be used as described previously (Grodberg, J. et al., (1989) op. cit.).

### Effect of Pla Inhibitors on Sensitivity of Bacterial Growth to Inhibition by CAMPs

To measure the effectiveness of Pla inhibitors at the cellular level, an assessment was made of the sensitivity of the growth of Y. pestis, S. typhimurium, and E. coli strains to the CAMPs LL-37, C18G [NH₂-ALYKKLLKKLLKSAKKLG (SEQ ID NO: 2)] (Darveau, R. P., et al., J. Clin. Invest. 90: 447-55 (1992)), and protamine, respectively, in the presence and absence of Pla inhibitors. These assays have been reported previously (Galvan, E. M., et al., (2008) op. cit.; Guina, T., et al., (2000) op. cit.; Stumpe, S. et al., (1998) op. cit.). Briefly, MIC determinations are made in the presence and absence of Pla inhibitors according to the CLSI guidelines (formerly NCCLS) (NCCLS, Methods for dilution antimicrobial susceptibility tests for bacteria that grow aerobically, Approved standard M7-A6, NCCLS, Wayne, Pa. (2003)). Increased sensitivity to growth inhibition by the CAMP (decreased MIC) in the presence of a Pla inhibitor indicates that the compound is inhibiting the proteolysis of CAMP. If direct MIC measurements are not sensitive enough, the CAMP may be preincubated with omptin-producing cells+/−added compound for various times (to provide sufficient time for proteolysis), followed by spin-down of the cells, and testing the supernatant for growth inhibitory effects on the appropriate strain. Maintenance of growth inhibitory effects only for preparations containing the Pla inhibitor will indicate that it functions to block proteolysis of the CAMP.

### Human Protease Counter-Screens

We examined the effect of confirmed Pla inhibitors on the plasminogen activator activity of the two human serine proteases, tissue plasminogen activator (tPA) and urokinase type plasminogen activator (uPA) in order to eliminate hits that do not exhibit species specificity for plasminogen activator inhibition. Human recombinant tPA and uPA (cat. Nos. T5600-30 and U2605-01, US Biological, Inc.) were assayed for activation of Glu-Plg using the chromogenic substrate S2251 to measure the plasmin produced (in the same manner as the Pla assay/screen). The concentration-dependence of confirmed Pla inhibitors was examined in these assays (IC₅₀) to determine the selectivity of Pla inhibition. Chromogenic substrates (Dalpharma, Inc., West Chester, Ohio) were used to test the Pla inhibitors for inhibition of a range of human serine proteases involved in the coagulation and fibrinoloysis pathways to ensure specificity and non-toxicity.

Pla inhibitors that inhibit either of two human aspartyl proteases, cathepsin D and E, which play roles in the normal physiological degradation of proteins and are implicated in the regulation of apoptosis (Tsukuba, T., K. et al., Mol. Cells, 10: 601-11 (2000), were eliminated. Recombinant human cathepsin D (cat. No. 1014-AS-010) and E (cat. No. 1294-AS-010) were purchased from R&D Systems. A fluorometric assay was used for determination of cathepsin D and E activity in the presence and absence of a range of confirmed hit concentrations, as described by Yasuda et al. (Yasuda, Y., T. et al., J. Biochem., 125:1137-43 (1999)) with an internally quenched fluorogenic peptide 7-Methoxycoumarin-4-Acetyl-GKPILFFRLK(DNP)-D-Arg-amide (SEQ ID NO: 3) (Sigma cat. No. M0938) as substrate in a 96-well plate. Cleavage at the Lys-Pro bond of the substrate by the cathepsins releases AMC and generates fluorescence (excitation at 360 nm; emission at 460 nm).

### Cell Invasion Assay

It is known that Y. pestis cells can invade both phagocytic and non-phagocytic cells, and under some circumstances replicate intracellularly (Cowan, C., et al., Infect. Immun., 68: 4523-30 (2000); Pujol, C., Proc. Natl. Acad. Sci. USA 102: 12909-14 (2005)). Y. pestis grown at 30° readily invades non-phagocytic cells, and Pla is required for efficient invasion (Lahteenmaki, K., M. et al., FEBS Lett., 504: 69-72 (2001)). We have recently shown that wild-type Y. pestis grown at 37° C. will also do so if exposure to atmospheric O₂ is sufficient, and also found that while Pla activity is required for invasion of some cell lines, for others Pla active site mutants will also promote invasion (Pouliot et al., in preparation). This implies that in some cases Pla may simply provide binding to a receptor that induces uptake. So-called “gentamicin (Gm) protection assays” are widely used to quantify bacterial invasion of mammalian cells because this antibiotic is very ineffective against intracellular bacteria (Cowan, C., et al., (2000) op. cit.). Accordingly, we may determine the ability of confirmed hits to block invasion of mammalian cells by Pla-producing Y. pestis using the standard Gm-protection protocol (Cowan, C., et al., (2000) op. cit.). Briefly, cells are first exposed to the bacteria at an MOI of 10 for 1 hr in the presence and absence of a range of confirmed hit concentrations, treated with Gm to kill residual extracellular bacteria, washed extensively, supplied with fresh media and incubated for an additional hour, washed again, subjected to osmotic lysis, and the lysate plated to determine the number of intracellular bacteria. These assays are conducted with invasive Y. pestis grown at both 30° and 37° C., and in both non-phagocytic cells (WI26 human derived lung epithelial cells) and a phagocytic human monocyte line (THP-1).

### Results

A schematic representation of the progression of compounds through the primary screen and the secondary confirmation and validation assays is depicted in FIG. 5. The 14 validated, non-cytotoxic inhibitors identified from the HTS of 109,000 compounds screened to date were categorized into three chemotypes and three singletons. An aromatic sulfonamide chemotype consisted of the largest number of members, and also contained some of the most potent inhibitors. Four aromatic sulfonamide screening hits are shown below (Compounds I-4). Table 2 below shows the results from the secondary assays demonstrating their specificity and low cytotoxicity.

TABLE 2
Compound 1

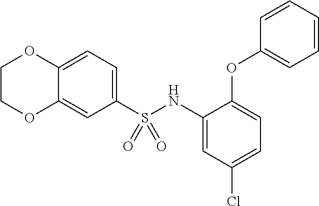

Compound 2

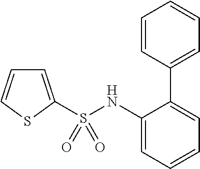

Compound 3

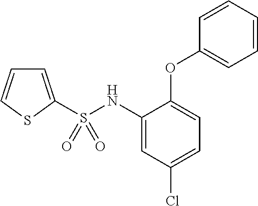

Compound 4

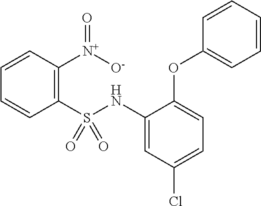

Qualification of Inhibitors of Y. pestis Pla:
Specificity of Inhibitory Activity vs. Pla Compared to Inhibitory activity vs.
Plasmin, tPA, uPA, Cathepsin D, Cathepsin E, and HIV-1 protease

| | Compound | Compound | Compound | Compound |
|---|---|---|---|---|
| | 1 | 2 | 3 | 4 |
| Plasmin Z-Score at | 4.9 | 1.8 | 2.5 | 1.4 |
| 50 μM^{a} | | | | |
| tPA Z-Score at 50 μM^{a} | −0.3 | 0.5 | 0.3 | 0.4 |
| uPA Z-Score at 50 μM^{a} | 0.5 | 0.5 | 0.5 | 1 |
| Cathepsin-D Z-Score | −0.2 | −1.6 | 0 | 0.6 |
| at 50 μM^{a} | | | | |
| Cathepsin-E Z-Score | −0.1 | −0.3 | 0.4 | 0.9 |
| at 50 μM^{a} | | | | |
| HIV-1 Protease | 10 | 2 | 13 | 11 |
| % inhibition at 50 μM^{b} | | | | |
| Pla IC₅₀ (μM)^{c} | 4.25 | 4.3 | 1 | 9.9 |
| Pla CC₅₀ (μM)^{d} | >100 | 62 | 49 | 47 |

^{a}Inhibition as measured by a z-score (number of standard deviations the enzymatic activity falls below the DMSO non-inhibitory control)
^{b}Inhibition as measured as a % of the enzymatic activity of a DMSO non-inhibitory control
^{c}Concentration of inhibitor that provides 50% inhibition in a concentration-dependency curve
^{d}Concentration of inhibitor that is cytotoxic to 50% of HeLa cells in serum-free culture

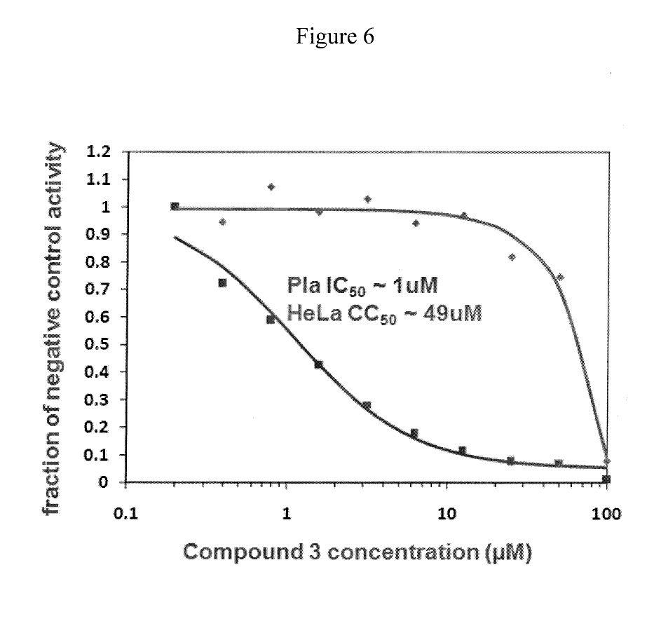

A graphical comparison of the potency (IC₅₀) and cytotoxicity (CC₅₀) of the most potent of the aromatic sulfonamides (Compound 3) is shown in FIG. 6. The selectivity ratio (CC₅₀/IC₅₀) is nearly 50-fold. Several analogs of the aromatic sulfonamide primary hits were tested in order to determine the relationship between structure and activity (see, Table 3).

TABLE 3

| | | IC₅₀ |
|---|---|---|
| CMPD | Structure | (μM) |
| A | 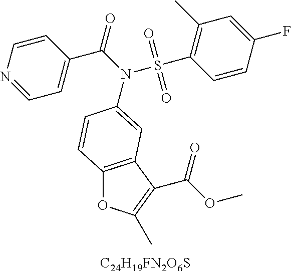 | 6.6 |
| B | 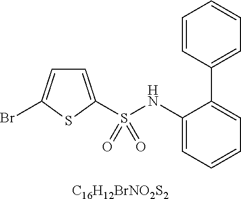 | 8.9 |
| C | 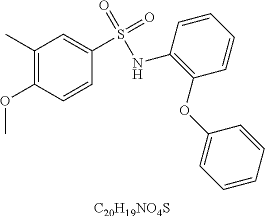 | 22.0 |
| D | 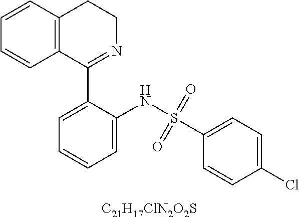 | 35.8 |
| E | 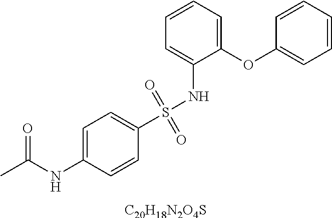 | 50.4 |
| F | 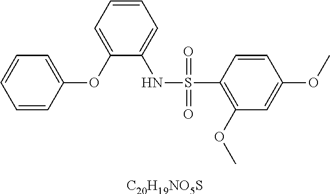 | 52.3 |
| G | 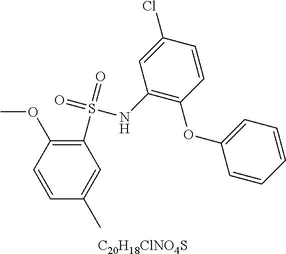 | 84.2 |
| H | 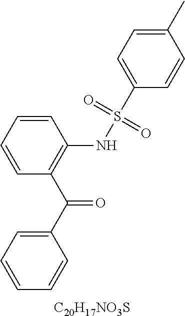 | 100.0 |
| I | 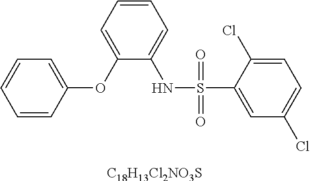 | 139.2 |

Consideration of the foregoing data defined a new group of compounds of related structure that are useful as bacterial omptin protease inhibitor compounds, and particularly, Yersinia pestis plasminogen activator (Pia) inhibitor compounds, and have potency and/or toxicity profiles that make them candidates for use as therapeutic agents. The new family of inhibitor compounds can be described by the Formula I:

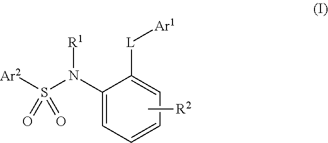

wherein:

L is a linker that is a direct bond or one of the following:

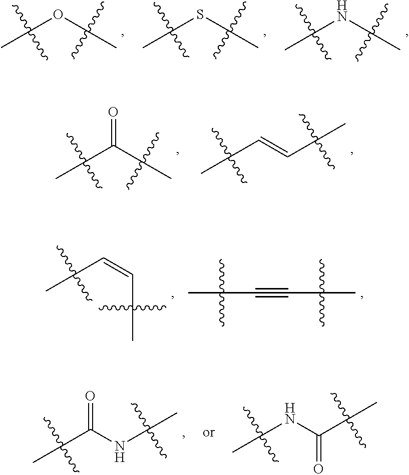

Ar¹ is a monovalent aryl or heteroaryl, cycloalkyl or heterocycloalkyl moiety which may be unsubstituted or substituted by up to 5 substituents selected from the group consisting of: halo, amino, amidino, guanidino, alkyl, haloalkyl, alkenyl, alkynyl, cycloalkyl, heterocycloalkyl, aryl, heteroaryl, hydroxy, alkoxy, aryloxy, heteroaryloxy, acyl, alkoxycarbonyl, aryloxycarbonyl, amino, substituted amino, acylamino, amido, sulfonamido, mercapto, alkylthio, arylthio, hydroxamate, thioacyl, alkylsulfonyl, or aminosulfonyl;

Ar² is a monovalent aryl or heteroaryl, moiety which may be unsubstituted or substituted by up to 5 substituents selected from the group consisting of: halo, amino, amidino, guanidino, alkyl, haloalkyl, alkenyl, alkynyl, cycloalkyl, heterocycloalkyl, aryl, heteroaryl, hydroxy, alkoxy, aryloxy, heteroaryloxy, acyl, carboxy, alkoxycarbonyl, aryloxycarbonyl, amino, substituted amino, acylamino, amido, sulfonamido, mercapto, alkylthio, arylthio, hydroxamate, thioacyl, alkylsulfonyl, or aminosulfonyl;

R¹ is a hydrogen or a monovalent alkyl, haloalkyl, alkenyl, alkynyl, cycloalkyl, heterocycloalkyl, or acyl moiety; and

R² represents a single or multiple substituents selected from the group consisting of: halo, amino, amidino, guanidino, alkyl, haloalkyl, alkenyl, alkynyl, cycloalkyl, heterocycloalkyl, aryl, heteroaryl, hydroxy, alkoxy, aryloxy, heteroaryloxy, acyl, carboxy, alkoxycarbonyl, aryloxycarbonyl, amino, substituted amino, acylamino, amido, sulfonamido, mercapto, alkylthio, arylthio, hydroxamate, thioacyl, alkylsulfonyl, or aminosulfonyl, located at the 3-, 4-, 5-, or 6-position of the phenyl ring;

and pharmaceutically acceptable salts thereof.

The compounds identified above are candidates for development as antibacterial agents, and particularly for the prevention and treatment of Y. pestis infection. The compounds may also be useful as molecular probes for the study of inhibition of bacterial omptin proteases.

All publications, patent applications, patents, and other documents cited herein are incorporated by reference in their entirety. Obvious variations to the disclosed compounds and alternative embodiments of the invention will be apparent to those skilled in the art in view of the foregoing disclosure. All such obvious variants and alternatives are considered to be within the scope of the invention as described herein.

We claim:

1. An isolated bacterial omptin protease inhibitor compound having the Formula (I): 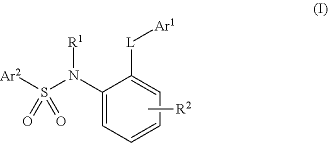
wherein:
L is a linker that is a direct bond or one of the following: 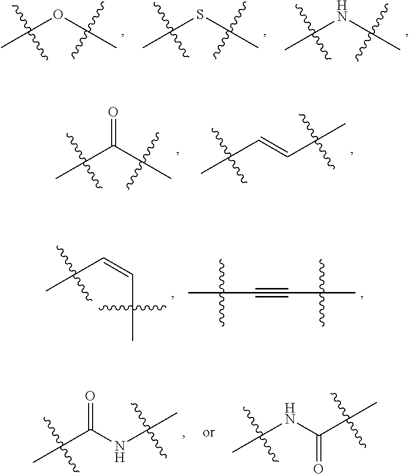
Ar¹ is a monovalent aryl or heteroaryl, cycloalkyl or heterocycloalkyl moiety which may be unsubstituted or substituted by up to 5 substituents selected from the group consisting of: halo, amino, amidino, guanidino, alkyl, haloalkyl, alkenyl, alkynyl, cycloalkyl, heterocycloalkyl, aryl, heteroaryl, hydroxy, alkoxy, aryloxy, heteroaryloxy, acyl, alkoxycarbonyl, aryloxycarbonyl, amino, substituted amino, acylamino, amido, sulfonamido, mercapto, alkylthio, arylthio, hydroxamate, thioacyl, alkylsulfonyl, or aminosulfonyl;
Ar² is a monovalent aryl or heteroaryl, moiety which may be unsubstituted or substituted by up to 5 substituents selected from the group consisting of: halo, amino, amidino, guanidino, alkyl, haloalkyl, alkenyl, alkynyl, cycloalkyl, heterocycloalkyl, aryl, heteroaryl, hydroxy, alkoxy, aryloxy, heteroaryloxy, acyl, carboxy, alkoxycarbonyl, aryloxycarbonyl, amino, substituted amino, acylamino, amido, sulfonamido, mercapto, alkylthio, arylthio, hydroxamate, thioacyl, alkylsulfonyl, or aminosulfonyl;
R¹ is a hydrogen or a monovalent alkyl, haloalkyl, alkenyl, alkynyl, cycloalkyl, heterocycloalkyl, or acyl moiety; and
R² represents a single or multiple substituents selected from the group consisting of: halo, amino, amidino, guanidino, alkyl, haloalkyl, alkenyl, alkynyl, cycloalkyl, heterocycloalkyl, aryl, heteroaryl, hydroxy, alkoxy, aryloxy, heteroaryloxy, acyl, carboxy, alkoxycarbonyl, aryloxycarbonyl, amino, substituted amino, acylamino, amido, sulfonamido, mercapto, alkylthio, arylthio, hydroxamate, thioacyl, alkylsulfonyl, or aminosulfonyl, located at the 3-, 4-, 5-, or 6-position of the phenyl ring;
and pharmaceutically acceptable salts thereof.

2. The isolated bacterial omptin protease inhibitor of claim 1, wherein the bacterial omptin protease is from bacterium selected from the group consisting of: Yersinia pestis, Enterobacter cloacae, Escherichia coli, Escherichia coli (EPEC), Klebsiella oxytoca, Klebsiella pneumoniae, Salmonella ssp., and Shigella ssp.

3. The isolated bacterial omptin protease inhibitor of claim 2, wherein the bacterial omptin protease is from Y. pestis.

4. The isolated bacterial omptin protease inhibitor of claim 3, wherein said compound inhibits Yersinia pestis plasminogen activator (Pla).

5. The isolated Y. pestis Pla inhibitor compound of claim 4, wherein said inhibitor compound has an IC₅₀ of less than 50 μM.

6. The isolated Y. pestis Pla inhibitor compound of claim 5, wherein said inhibitor compound has an IC₅₀ of less than 25 μM.

7. The isolated Y. pestis Pla inhibitor compound of claim 6, wherein said inhibitor compound has a CC₅₀ value of greater than or equal to 50 μM.

8. An isolated Y. pestis plasminogen activator inhibitor compound of the formula: 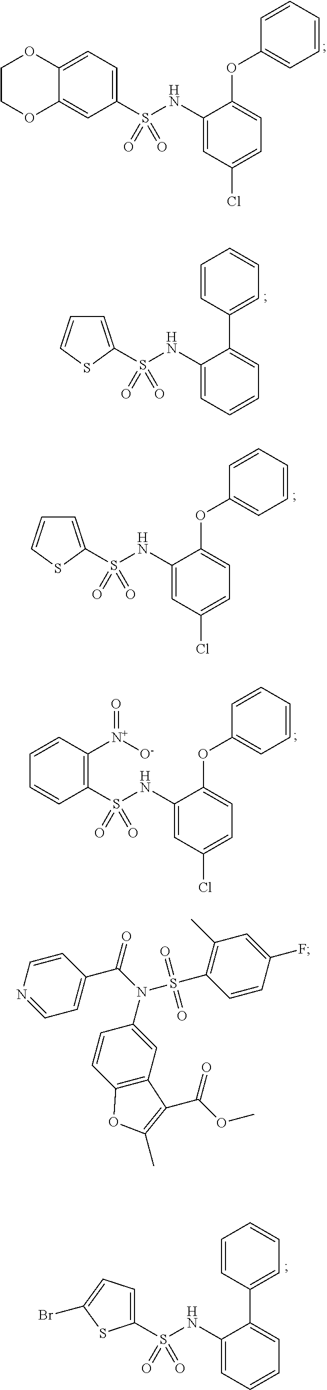 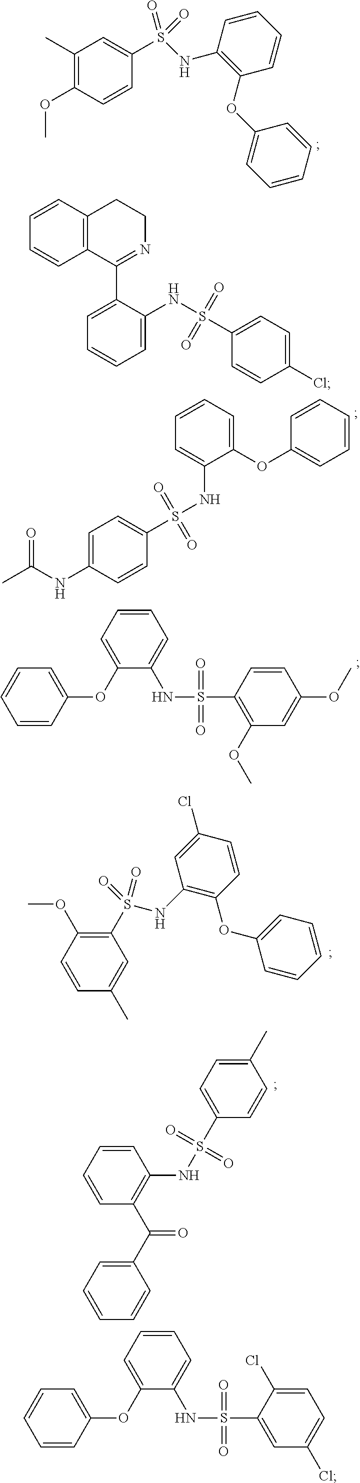
and pharmaceutically acceptable salts thereof.

9. A pharmaceutical composition comprising one or more Y. pestis plasminogen activator inhibitor compounds according to claim 4 or claim 8 and a pharmaceutically acceptable carrier or excipient.

10. A method for treating an individual infected with or exposed to Y. pestis comprising administering to said individual, as an active ingredient, a compound or composition according to claim 1 or claim 8.

11. The method according to claim 11, wherein said individual is a human.

12. The method according to claim 11, further comprising administering an additional active ingredient in conjunction with said Y. pestis plasminogen activator inhibitor compound, said additional active ingredient being selected from the group consisting of an antibiotic, an antibody, an antiviral agent, an anticancer agent, an analgesic, an immunostimulatory agent, a natural, synthetic or semi-synthetic hormone, a central nervous system stimulant, an antiemetic agent, an anti-histamine, an erythropoietin, a complement stimulating agent, a sedative, a muscle relaxant agent, an anesthetic agent, an anticonvulsive agent, an antidepressant, an antipsychotic agent, a type three secretion system (T3SS) inhibitor, and combinations thereof.

13. A method of inhibiting and/or reducing dissemination of bacterium in a mammal, said method comprising administering to said mammal, as an active ingredient, a compound or composition according to claim 1 or claim 8.

14. The method according to claim 13, wherein said mammal is a human.

15. The method according to claim 14, wherein said bacterium is selected from the group consisting of Yersinia pestis, Enterobacter cloacae, Escherichia coli, Escherichia coli (EPEC), Klebsiella oxytoca, Klebsiella pneumoniae, Salmonella ssp., and Shigella ssp.

16. The method according to claim 15, wherein said bacterium is Yersinia pestis.
